(19) United States
(12) Patent Application Publication
(10) Pub. No.: US 2026/0207945 A1
(43) Pub. Date: Jul. 23, 2026

(54) METHOD AND APPARATUS FOR CARDIAC EVENT SIGNAL SENSING

(71) Applicant: Medtronic, Inc., Minneapolis, MN (US)

(72) Inventors: Ronson Lok YONG, Shoreview, MN (US); Michael L. HUDZIAK, Stillwater, MN (US)

(21) Appl. No.: 19/141,293

(22) PCT Filed: Nov. 21, 2023
(86) PCT No.: PCT/IB2023/061732

§ 371 Date: Jun. 19, 2025

Related U.S. Application Data
(60) Provisional application No. 63/476,626, filed on Dec. 21, 2022.

(51) Int. Cl. A61N 1/365

(52) U.S. Cl. CPC A61N 1/365

(57) ABSTRACT

A medical device is configured to sense a cardiac signal and, without rectifying the cardiac signal, apply a first cardiac event sensing threshold amplitude having a first polarity to the cardiac signal. In response to the cardiac signal crossing the first sensing threshold amplitude, the medical device may apply to the cardiac signal a second sensing threshold amplitude that has a second polarity opposite the first polarity. The medical device may determine when a requirement relating to the second sensing threshold amplitude is met by the first cardiac signal for use in confirming a sensed cardiac event signal.

This application claims the benefit of U.S. Provisional Patent Application Ser. No. 63/476,626, filed Dec. 21, 2022, the entire content of which is incorporated herein by reference.

### TECHNICAL FIELD

This disclosure relates to a medical device configured to sense cardiac event signals and discriminate between cardiac event signals arising from atrial and ventricular heart chambers.

### BACKGROUND

During normal sinus rhythm (NSR), the heartbeat is regulated by electrical signals produced by the sino-atrial (SA) node located in the right atrial wall. Each atrial depolarization signal produced by the SA node spreads across the atria, causing the depolarization and contraction of the atria, and arrives at the atrioventricular (AV) node. The AV node responds by propagating a ventricular depolarization signal through the bundle of His of the ventricular septum and thereafter to the bundle branches and the Purkinje muscle fibers of the right and left ventricles, sometimes referred to as the “His-Purkinje system.”

Patients with a conduction system abnormality, e.g., SA node dysfunction or poor AV node conduction, bundle branch block, or other conduction abnormalities of the heart, may receive a pacemaker to restore a more normal heart rhythm. A single chamber pacemaker coupled to a transvenous lead carrying electrodes positioned in the right atrium may provide atrial pacing to treat a patient having SA node dysfunction. When the AV node and His-Purkinje system conduction is functioning normally, single chamber atrial pacing may sufficiently correct the heart rhythm. The pacing-evoked atrial depolarizations may be conducted normally to the ventricles via the AV node and the His-Purkinje system maintaining normal AV synchrony. Some patients, however, may experience conduction abnormalities of the AV node, e.g., partial or complete AV block, and/or conduction abnormalities of the His-Purkinje system. AV block may be intermittent and may evolve over time. For example, in the presence of AV block, atrial depolarizations may not be conducted to the ventricles on every atrial cycle or may be conducted but at a prolonged AV conduction time resulting in poor AV synchrony in the native heart rhythm. In this case, the patient may require a single chamber ventricular pacemaker or a dual chamber pacemaker.

A dual chamber pacemaker may be implanted in some patients to pace both the atrial and ventricular chambers and thereby maintain AV synchrony. The dual chamber pacemaker may be coupled to a transvenous atrial lead and a transvenous ventricular lead, for placing electrodes for sensing and pacing in both the atrial and ventricular chambers. The pacemaker itself can be implanted in a subcutaneous pocket with the transvenous leads tunneled to the subcutaneous pocket.

Intracardiac pacemakers have been introduced or proposed for implantation entirely within a patient's heart eliminating the need for transvenous leads. For example, one or more intracardiac pacemakers may provide sensing and pacing from within a heart chamber of a patient having a conduction abnormality to provide single or dual chamber pacing to promote a more normal heart rhythm.

### SUMMARY

The techniques of this disclosure generally relate to a medical device configured to sense cardiac event signals attendant to myocardial depolarizations (and/or repolarizations). The medical device may receive one or more cardiac electrical signals from electrodes implanted in or on a heart chamber. The medical device can be configured to sense cardiac event signals from the received cardiac electrical signal(s) in a manner that distinguishes between P-waves arising from the atria and R-waves arriving from the ventricles. The medical device may be capable of generating cardiac pacing pulses. The timing of generated cardiac pacing pulses may be controlled by the medical device based on the sensed cardiac event signals.

A medical device operating according to the techniques disclosed herein may apply at least two different sensing thresholds to a non-rectified cardiac signal sensed by sensing circuitry of the medical device. The two sensing thresholds may be defined to have opposing polarities and may have the same or different absolute value amplitude. A cardiac event signal, e.g., a P-wave or an R-wave, may be sensed when a non-rectified, sensed cardiac signal meets cardiac event sensing criteria based on at least two opposing polarity sensing threshold requirements. For example, cardiac event sensing criteria may be met when a cardiac electrical signal crosses a first polarity sensing threshold and meets a second requirement relating to a second polarity sensing threshold having the opposite polarity of the first polarity sensing threshold. The second requirement may be to cross the second polarity sensing threshold in some examples. In other examples the second requirement may be to not cross the second polarity sensing threshold. The sensed cardiac signal may be confirmed as a sensed cardiac event signal when the second requirement is met within a confirmation time window from the first polarity sensing threshold crossing by the cardiac signal. In various examples, an atrial event signal is sensed and confirmed by sensing circuitry of the medical device based on a first polarity atrial sensing threshold crossing and a second requirement relating to a second polarity atrial sensing threshold being met. Additionally or alternatively, a ventricular event signal may be sensed and confirmed by sensing circuitry of the medical device based on a first polarity ventricular sensing threshold crossing and a second requirement relating to a second polarity ventricular sensing threshold being met.

In one example, the disclosure provides a medical device including sensing circuitry configured to sense a cardiac signal and, without rectifying the cardiac signal, apply a first sensing threshold amplitude having a first polarity to the cardiac signal. In response to the cardiac signal crossing the first sensing threshold amplitude, the sensing circuitry may apply a second sensing threshold amplitude to the cardiac signal where the second sensing threshold amplitude has a second polarity opposite the first polarity. The sensing circuitry may be further configured to determine when a requirement relating to the second sensing threshold amplitude is met by the cardiac signal and confirm a sensed cardiac event signal corresponding to a depolarization of a heart chamber in response to at least the cardiac signal crossing the first sensing threshold amplitude and meeting the requirement relating to the second sensing threshold amplitude. The medical device may further include control circuitry configured to start a timing control interval in response to the confirmed sensed cardiac event signal.

In another example, the disclosure provides a method including sensing a cardiac signal and, without rectifying the cardiac signal, applying a first sensing threshold amplitude having a first polarity to the cardiac signal. In response to the cardiac signal crossing the first sensing threshold amplitude, the method may include applying a second sensing threshold amplitude to the cardiac signal where the second sensing threshold amplitude has a second polarity opposite the first polarity. The method may further include determining when a requirement relating to the second sensing threshold amplitude is met by the cardiac signal and confirming a sensed cardiac event signal corresponding to a depolarization of a first heart chamber in response to at least the cardiac signal crossing the first threshold amplitude and meeting the requirement relating to the second sensing threshold amplitude. The method may further include starting a first timing control interval in response to the confirmed sensed first cardiac event signal.

In another example, the disclosure provides a non-transitory, computer-readable storage medium comprising a set of instructions which, when executed by processing circuitry of a medical device, cause the medical device to sense a cardiac signal and, without rectifying the cardiac signal, apply a first cardiac event sensing threshold amplitude having a first polarity to the cardiac signal. The instructions may further cause the medical device to, in response to the cardiac signal crossing the first sensing threshold amplitude, apply a second sensing threshold amplitude to the cardiac signal where the second sensing threshold amplitude has a second polarity opposite the first polarity. The instructions may further cause the medical device to determine when a requirement relating to the second sensing threshold amplitude is met by the cardiac signal. The instructions may further cause the medical device to confirm a sensed cardiac event signal corresponding to a depolarization of a heart chamber in response to at least the cardiac signal crossing the first threshold amplitude and meeting the requirement relating to the second sensing threshold amplitude. The instructions may further cause the medical device to start a timing control interval in response to the confirmed sensed first cardiac event signal.

Further disclosed herein is the subject matter of the following examples:

Example 1. A medical device comprising sensing circuitry configured to sense a first cardiac signal and, without rectification of the first cardiac signal, apply a first sensing threshold amplitude having a first polarity to the first cardiac signal. The sensing circuitry may be configured to, in response to the first cardiac signal crossing the first sensing threshold amplitude, apply a second sensing threshold amplitude to the first cardiac signal, the second sensing threshold amplitude having a second polarity opposite the first polarity. The sensing circuitry may be further configured to determine when a requirement relating to the second sensing threshold amplitude is met by the first cardiac signal and confirm a sensed first cardiac event signal corresponding to a depolarization of a first heart chamber in response to at least the first cardiac signal crossing the first sensing threshold amplitude and meeting the requirement relating to the second sensing threshold amplitude. The medical device may further include control circuitry configured to start a first timing control interval in response to the confirmed sensed first cardiac event signal.

Example 2. The medical device of example 1 wherein the sensing circuitry is further configured to start a first confirmation window in response to the first cardiac signal crossing the first sensing threshold amplitude and determine that the requirement relating to the second sensing threshold amplitude is met by the first cardiac signal being sensed during the first confirmation window.

Example 3. The medical device of any of examples 1-2 wherein the sensing circuitry is configured to determine that the requirement relating to the second sensing threshold amplitude is met by the first cardiac signal by determining that the first cardiac signal does not cross the second sensing threshold amplitude.

Example 4. The medical device of any of examples 1-3 wherein the sensing circuitry is further configured to start a first confirmation window in response to the first cardiac signal crossing the first sensing threshold amplitude, sense a second cardiac signal, apply a third sensing threshold amplitude to the second cardiac signal, start a second confirmation window in response to the second cardiac signal crossing the third sensing threshold amplitude. The sensing circuitry may determine that the second confirmation window is running during a portion of the first confirmation window and, in response to the second confirmation window running during a portion of the first confirmation window, withhold confirming the sensed first cardiac event signal.

Example 5. The medical device of example 4 wherein the sensing circuitry is further configured to apply a fourth sensing threshold amplitude to the second cardiac signal sensed during the second confirmation window where the fourth sensing threshold amplitude has an opposite polarity from the third sensing threshold amplitude. The sensing circuitry may be further configured to determine that the second cardiac signal meets a requirement relating to the fourth sensing threshold amplitude having a different polarity than the third sensing threshold amplitude and confirm a sensed second cardiac event signal corresponding to a depolarization of a second heart chamber different than the first heart chamber in response to the second cardiac signal crossing the third sensing threshold amplitude and meeting the requirement relating to the fourth sensing threshold amplitude. The control circuitry may be further configured to start a second timing control interval in response to the confirmed sensed second cardiac event signal.

Example 6. The medical device of example 5 wherein the sensing circuitry is further configured to determine that the second cardiac signal meets the requirement relating to the fourth sensing threshold amplitude by determining that the second cardiac signal crosses the fourth sensing threshold amplitude.

Example 7. The medical device of any of examples 5-6 wherein the control circuit is configured to start the second timing control interval by starting a pacing interval. The medical device may further include a therapy delivery circuit configured to generate a pacing pulse upon expiration of the pacing interval.

Example 8. The medical device of any of examples 4-7 wherein the sensing circuitry is further configured to determine that the second cardiac signal sensed during the second confirmation window does not meet the requirement relating to the fourth sensing threshold amplitude and confirm the sensed first cardiac event signal when the second cardiac signal does not meet the requirement relating to the fourth sensing threshold amplitude.

Example 9. The medical device of any of examples 1-8 wherein the sensing circuitry is further configured to sense a second cardiac signal, start a first confirmation window in response to the first cardiac signal crossing the first sensing threshold amplitude, determine a first feature of the first cardiac signal sensed during the first confirmation window, determine a second feature of the second cardiac signal sensed during the first confirmation window and confirm the sensed first cardiac event signal based on a comparison between the first feature and the second feature.

Example 10. The medical device of example 9 wherein the sensing circuitry is further configured to determine the first feature as one of a first peak amplitude or a first peak slope of the first cardiac signal and determine the second feature as one of a second peak amplitude or a second peak slope of the second cardiac signal.

Example 11. The medical device of any of examples 1-3 wherein the sensing circuitry is further configured to, in response to the first cardiac signal crossing the first sensing threshold amplitude, start a first confirmation window and apply a third sensing threshold amplitude to the first cardiac signal during the first confirmation window, start a second confirmation window upon expiration of the first confirmation window and apply the second sensing threshold amplitude having the second polarity opposite the first polarity during the second confirmation window. The sensing circuitry may be configured to determine a first amplitude zone relative to the first sensing threshold amplitude and the third sensing threshold amplitude, determine a second amplitude zone relative to the second sensing threshold amplitude and determine when the requirement relating to the second sensing threshold amplitude is met based on the first amplitude zone and the second amplitude zone.

Example 12. The medical device of example 11 wherein the sensing circuitry is further configured to select the second sensing threshold amplitude that is applied during the second confirmation window based on the first amplitude zone.

Example 13. The medical device of any of examples 11-12 wherein the sensing circuitry is further configured to, in response to determining that the requirement relating to the second sensing threshold amplitude is not met, confirm a sensed second cardiac event signal corresponding to a second heart chamber based on the first amplitude zone and the second amplitude zone.

Example 14. The medical device of any of examples 11-13 wherein the sensing circuitry is further configured to sense a second cardiac signal, determine an indeterminate waveform when the requirement relating to the second sensing threshold amplitude is not met based on the first amplitude zone and the second amplitude zone and identify a sensed waveform based on the second cardiac signal when the indeterminate waveform is determined.

Example 15. The medical device of any of examples 1-14 wherein the control circuitry is further configured to start the first timing control interval by starting at least one of: a blanking period; a refractory period; and a pacing interval.

Example 16. The medical device of example 15 wherein the sensing circuitry is further configured to confirm the sensed first cardiac event as being a ventricular event signal and the control circuitry is further configured to start the first timing control interval by starting at least one of: a ventricular blanking period; a ventricular refractory period; a post-ventricular atrial blanking period; and a ventricular pacing interval.

Example 17. The medical device of example 15 wherein the sensing circuitry is further configured to confirm the sensed first cardiac event as an atrial event signal and the control circuitry is further configured to start the first timing control interval by starting at least one of: an atrial blanking period; an atrial refractory period; a post-atrial ventricular blanking period; an atrial pacing interval; and an atrioventricular pacing interval.

Example 18. The medical device of example 15 wherein the sensing circuitry is further configured to confirm the sensed first cardiac event as an atrial event signal, and the control circuitry is configured to start the first timing control interval by starting an atrioventricular pacing interval. The medical device may further include a therapy delivery circuit configured to generate a ventricular pacing pulse upon expiration of the atrioventricular pacing interval.

Example 19. The medical device of any of examples 1-15 wherein the control circuitry is further configured to start the first timing control interval by starting at least a first pacing interval in response to the confirmed sensed first cardiac event signal and determine that the first pacing interval is expired. The medical device may further include a therapy delivery circuit configured to generate a pacing pulse in response to the first pacing interval being expired.

Example 20. The medical device of any of examples 1-19 further including a housing enclosing the sensing circuitry and the control circuitry and at least one leadless, housing-based tissue piercing electrode coupled to the sensing circuitry for sensing the first cardiac signal.

Example 21. The medical device of any of examples 1-20 further including a therapy delivery circuit coupled to at least one leadless, housing-based tissue-piercing electrode for delivering a pacing pulse to a conduction system of a patient's heart upon expiration of the first timing control interval.

Example 22. The medical device of any of examples 1-21 wherein the sensing circuitry is further configured to apply a third sensing threshold amplitude to the first cardiac signal and, in response to the first cardiac signal crossing the third sensing threshold amplitude, apply a fourth sensing threshold amplitude to the first cardiac signal, the fourth sensing threshold amplitude having a polarity opposite the third sensing threshold amplitude. The sensing circuitry may be further configured to determine that a requirement relating to the third sensing threshold amplitude is met by the first cardiac signal and confirm a sensed second cardiac event signal corresponding to a depolarization of a second heart chamber in response to at least the first cardiac signal crossing the third sensing threshold amplitude and meeting the requirement relating to the fourth sensing threshold amplitude. The control circuitry may be further configured to start a second timing control interval in response to the confirmed sensed second cardiac event signal.

Example 23. The medical device of any of examples 1-22 wherein the control circuitry is further configured to start the first timing control interval by starting at least a first pacing interval in response to the confirmed sensed first cardiac event signal and determine that the first pacing interval is expired. The medical device may further include a therapy delivery circuit configured to generate a pacing pulse in response to the first pacing interval being expired.

Example 24. The medical device of example 23 wherein the sensing circuitry is further configured to confirm the sensed first cardiac event as an atrial event signal and the control circuitry is configured to start at least the first pacing interval by starting an atrioventricular pacing interval. The therapy delivery circuit may be configured to generate the pacing pulse by generating a ventricular pacing pulse upon expiration of the atrioventricular pacing interval.

Example 25. The medical device of any of examples 23-24 further including a housing enclosing the sensing circuitry and the control circuitry and at least one leadless, housing-based tissue piercing electrode coupled to the therapy delivery circuit for delivering a pacing pulse to a conduction system of a patient's heart upon expiration of the first pacing interval.

Example 26. A method including sensing a first cardiac signal and, without rectifying the first cardiac signal, applying a first sensing threshold amplitude having a first polarity to the first cardiac signal. In response to the first cardiac signal crossing the first sensing threshold amplitude, the method may include applying a second sensing threshold amplitude to the first cardiac signal, the second sensing threshold amplitude having a second polarity opposite the first polarity. The method may further include determining when a requirement relating to the second sensing threshold amplitude is met by the first cardiac signal, confirming a sensed first cardiac event signal corresponding to a depolarization of a first heart chamber in response to at least the first cardiac signal crossing the first threshold amplitude and meeting the requirement relating to the second sensing threshold amplitude and starting a first timing control interval in response to the confirmed sensed first cardiac event signal.

Example 27. The method of example 26 further including starting a first confirmation window in response to the first cardiac signal crossing the first sensing threshold amplitude and determining that the requirement relating to the second sensing threshold amplitude is met by the first cardiac signal sensed during the first confirmation window.

Example 28. The method of any of examples 26-27 further including determining that the requirement relating to the second sensing threshold amplitude is met by the first cardiac signal by determining that the first cardiac signal does not cross the second sensing threshold amplitude.

Example 29. The method of any of examples 26-28 further including starting a first confirmation window in response to the first cardiac signal crossing the first sensing threshold amplitude, sensing a second cardiac signal and applying a third sensing threshold amplitude to the second cardiac signal. The method may further include starting a second confirmation window in response to the second cardiac signal crossing the third sensing threshold amplitude, determining that the second confirmation window is running during a portion of the first confirmation window and, in response to the second confirmation window running during a portion of the first confirmation window, withholding confirming the sensed first cardiac event signal.

Example 30. The method of example 29 further including applying a fourth sensing threshold amplitude to the second cardiac signal sensed during the second confirmation window, the fourth sensing threshold amplitude having an opposite polarity from the third sensing threshold amplitude. The method may further include determining that the second cardiac signal meets a requirement relating to the fourth sensing threshold amplitude having a different polarity than the third sensing threshold amplitude. The method may further include confirming a sensed second cardiac event signal corresponding to a depolarization of a second heart chamber different than the first heart chamber in response to the second cardiac signal crossing the third sensing threshold amplitude and meeting the requirement relating to the fourth sensing threshold amplitude. The method may include starting a second timing control interval in response to the confirmed sensed second cardiac event signal.

Example 31. The method of example 30 further including determining that the second cardiac signal meets the requirement relating to the fourth sensing threshold amplitude by determining that the second cardiac signal crosses the fourth sensing threshold amplitude.

Example 32. The method of any of examples 30-31 further including starting the second timing control interval by starting a pacing interval and generating a pacing pulse upon expiration of the pacing interval.

Example 33. The method of any of examples 30-32 further including determining that the second cardiac signal sensed during the second confirmation window does not meet the requirement relating to the fourth sensing threshold amplitude and confirming the sensed first cardiac event signal in response to the second cardiac signal not meeting the requirement relating to the fourth sensing threshold amplitude.

Example 34. The method of any of examples 26-33 further including sensing a second cardiac signal, starting a first confirmation window in response to the first cardiac signal crossing the first sensing threshold amplitude, determining a first feature of the first cardiac signal sensed during the first confirmation window and determining a second feature of the second cardiac signal sensed during the first confirmation window. The method may further include confirming the sensed first cardiac event signal based on a comparison between the first feature and the second feature.

Example 35. The method of example 34 further including determining the first feature as one of a first peak amplitude or a first peak slope of the first cardiac signal and determining the second feature as one of a second peak amplitude or a second peak slope of the second cardiac signal.

Example 36. The method of any of examples 26-29 further including, in response to the first cardiac signal crossing the first sensing threshold amplitude starting a first confirmation window, applying a third sensing threshold amplitude to the first cardiac signal during the first confirmation window, starting a second confirmation window upon expiration of the first confirmation window and applying the second sensing threshold amplitude having the second polarity opposite the first polarity during the second confirmation window. The method may further include determining a first amplitude zone relative to the first sensing threshold amplitude and the third sensing threshold amplitude, determining a second amplitude zone relative to the second sensing threshold amplitude; and determining when the requirement relating to the second sensing threshold amplitude is met based on the first amplitude zone and the second amplitude zone meeting.

Example 37. The method of example 36 further including selecting the second sensing threshold amplitude that is applied during the second confirmation window based on the first amplitude zone.

Example 38. The method of any of examples 36-37 further including in response to determining that the requirement relating to the second sensing threshold amplitude is not met confirming a sensed second cardiac event signal corresponding to a second heart chamber based on the first amplitude zone and the second amplitude zone.

Example 39. The method of any of examples 36-38 further including sensing a second cardiac signal, determining an indeterminate waveform when the requirement relating to the second sensing threshold amplitude is not met based on the first amplitude zone and the second amplitude zone and identifying a sensed waveform based on the second cardiac signal when the indeterminate waveform is determined.

Example 40. The method of any of examples 26-39 further including starting the first timing control interval by starting at least one of: a blanking period; a refractory period; and a pacing interval.

Example 41. The method of example 40 further including confirming the sensed first cardiac event as being a ventricular event signal and starting the first timing control interval by starting at least one of: a ventricular blanking period; a ventricular refractory period; a post-ventricular atrial blanking period; and a ventricular pacing interval.

Example 42. The method of example 40 further including confirming the sensed first cardiac event as an atrial event signal and starting the first timing control interval by starting at least one of: an atrial blanking period; an atrial refractory period; a post-atrial ventricular blanking period; an atrial pacing interval; and an atrioventricular pacing interval.

Example 43. The method of example 40 further including confirming the sensed first cardiac event as an atrial event signal, starting the first timing control interval by starting an atrioventricular pacing interval and generating a ventricular pacing pulse upon expiration of the atrioventricular pacing interval.

Example 44. The method of any of examples 26-40 further including starting the first timing control interval by starting at least a first pacing interval in response to the confirmed sensed first cardiac event signal, determining that the first pacing interval is expired and generating a pacing pulse in response to the first pacing interval being expired.

Example 45. The method of any of examples 26-44 further including sensing the first cardiac signal using at least one leadless, housing-based electrode.

Example 46. The method of any of examples 26-45 further including detecting an expiration of the first timing control interval and delivering a pacing pulse to a conduction system of a patient's heart via at least one leadless, housing-based tissue-piercing electrode upon expiration of the first timing control interval.

Example 47. The method of any of examples 26-46 further including applying a third sensing threshold amplitude to the first cardiac signal and, in response to the first cardiac signal crossing the third sensing threshold amplitude, applying a fourth sensing threshold amplitude to the first cardiac signal, the fourth sensing threshold amplitude having a polarity opposite the third sensing threshold amplitude. The method may further include determining that a requirement relating to the third sensing threshold amplitude is met by the first cardiac signal and confirming a sensed second cardiac event signal corresponding to a depolarization of a second heart chamber in response to at least the first cardiac signal crossing the third sensing threshold amplitude and meeting the requirement relating to the fourth sensing threshold amplitude. The method may further include starting a second timing control interval in response to the confirmed sensed second cardiac event signal.

Example 48. A non-transitory, computer-readable storage medium storing a set of instructions which, when executed by processing circuitry of a medical device, cause the medical device to sense a cardiac signal and, without rectifying the cardiac signal, apply a first cardiac event sensing threshold amplitude having a first polarity to the cardiac signal. In response to the cardiac signal crossing the first sensing threshold amplitude the instructions may further cause the device to apply a second sensing threshold amplitude to the cardiac signal, the second sensing threshold amplitude having a second polarity opposite the first polarity. The instructions may further cause the device to determine when a requirement relating to the second sensing threshold amplitude is met by the cardiac signal, confirm a sensed first cardiac event signal corresponding to a depolarization of a first heart chamber in response to at least the cardiac signal crossing the first threshold amplitude and meeting the requirement relating to the second sensing threshold amplitude, and start a timing control interval in response to the confirmed sensed first cardiac event signal.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram illustrating an implantable medical device (IMD) system that may be used to sense cardiac signals and provide cardiac pacing.

FIG. 2 is a conceptual diagram of the pacemaker shown in FIG. 1 according to one example.

FIG. 3 is a conceptual diagram of an example configuration of the pacemaker of FIG. 1 according to some examples.

FIG. 4 is a conceptual diagram of sensing circuitry that may be included in a pacemaker according to some examples.

FIG. 5A is a diagram of an atrial electrogram (EGM) signal, and FIG. 5B is a diagram of a ventricular EGM signal that may be received cardiac event detection circuitry included in a medical device for sensing P-waves and R-waves, respectively, according to some examples.

FIG. 6 is a flow chart of a method that can be performed by the pacemaker of FIG. 1 for sensing P-waves according to some examples.

FIG. 7 is a flow chart of a method that can be performed by the pacemaker of FIG. 1 for sensing R-waves according to some examples.

FIG. 8 is a flow chart of a method that may be performed by sensing and control circuitry of pacemaker for sensing cardiac event signals according to another example.

FIG. 9 is a diagram of example thresholds and time windows that may be applied to a sensed cardiac electrical signal for sensing and discriminating P-waves, R-waves and, if desired, T-waves, according to some examples.

FIG. 10 is a flow chart of a method for sensing cardiac event signals according to some examples.

FIG. 11 is a diagram of confirmation windows and sensing thresholds that may be applied to a cardiac electrical signal for sensing cardiac event signals according to some examples.

FIG. 12 is a conceptual diagram of variations in sensed cardiac electrical signals due to differences in inter-electrode spacing of a sensing electrode vector.

### DETAILED DESCRIPTION

In general, this disclosure describes a medical device and method for sensing cardiac event signals attendant to atrial depolarizations (e.g., P-waves) and ventricular depolarizations (e.g., R-waves) from a cardiac signal, e.g., a cardiac electrical signal such as an EGM signal or ECG signal. In some cases the cardiac event signal sensed using the disclosed techniques may be a T-wave attendant to ventricular myocardial repolarization. The term “cardiac event signal” as used herein refers to a waveform of a cardiac signal that corresponds to myocardial depolarization or repolarization that occurs during a heartbeat. A cardiac event signal, therefore, may be a P-wave, an R-wave or a T-wave. In some instances, the cardiac event signal may be a non-sinus event signal, e.g., an ectopic or other aberrantly conducted depolarization of cardiac tissue such as a premature atrial contraction or a premature ventricular contraction. The medical device may sense the cardiac event signal(s) for a variety of purposes such as determining a heart rate, detecting arrhythmia, and controlling cardiac electrical stimulation therapy such as bradycardia pacing or anti-tachycardia pacing (ATP) or delivering a cardioversion or defibrillation shock. Depending on the location of sensing electrodes used for sensing cardiac event signals, atrial P-waves and ventricular R-waves may be difficult to distinguish from each other. Techniques disclosed herein provide for cardiac event signal sensing that reliably senses and distinguishes P-waves and R-waves (and/or other cardiac event signals) in a medical device, which may be configured for single chamber or dual chamber sensing.

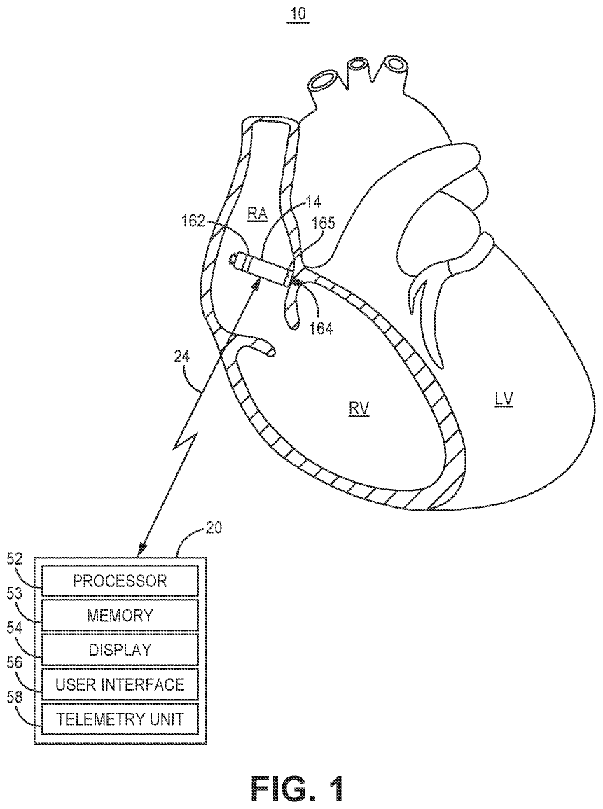

FIG. 1 is a conceptual diagram illustrating an implantable medical device (IMD) system 10 that may be used to sense cardiac signals and provide cardiac pacing. IMD system 10 is shown including a pacemaker 14, implanted within the right atrium (RA). In some examples, pacemaker 14 is a transcatheter, leadless pacemaker that can be implanted wholly within a heart chamber or on a heart chamber. Pacemaker 14 may be reduced in size compared to subcutaneously implanted pacemakers and may be generally cylindrical in shape to facilitate transvenous implantation via a delivery catheter. Pacemaker 14 may be a leadless pacemaker that includes electrodes carried on the pacemaker housing without requiring medical electrical leads extending from pacemaker 14 for sensing cardiac electrical signals and delivering cardiac pacing pulses.

Pacemaker 14 is configured for sensing atrial event signals, e.g., P-waves attendant to atrial depolarizations, and/or ventricular event signals, e.g., R-waves attendant to ventricular depolarizations, according to the techniques disclosed herein. Pacemaker 14 may be configured as a dual chamber pacemaker capable of sensing both atrial and ventricular event signals and delivering atrial pacing pulses and ventricular pacing pulses as needed based on the sensed atrial and/or ventricular event signals. In other examples, pacemaker 14 may be configured as a single chamber pacemaker capable of delivering only atrial pacing pulses or capable of delivering only ventricular pacing pulses and may be capable of single chamber (atrial or ventricular) sensing or dual chamber sensing of both atrial and ventricular event signals. In still other examples, pacemaker 14 may be configured to sense and pace a single heart chamber, atrial or ventricular, but may use the techniques disclosed herein for sensing cardiac event signals arising from a heart chamber, atrial or ventricular, distinct from cardiac event signals arising from a different heart chamber, ventricular or atrial.

In the example shown, pacemaker 14 is implanted in the RA for providing ventricular pacing from an atrial location. Pacemaker 14 may be configured for delivering ventricular pacing pulses via the heart's native conduction system and/or ventricular myocardium from a RA approach. For example, the distal end of pacemaker 14 may be positioned at the inferior end of the interatrial septum, beneath the AV node and near the tricuspid valve annulus to position a tip electrode 164 for advancement into the interatrial septum toward the His bundle of the native His-Purkinje conduction system. A target entry site for electrode 164 may correspond to or lie within the Triangle of Koch in some examples for achieving ventricular pacing from an atrial implant location of pacemaker 14. Ventricular pacing of the conduction system of the heart, e.g., at a His bundle pacing site, may be achieved from this example implant location.

A second electrode, e.g., a ring electrode 162 and/or ring electrode 165, may be spaced proximally from the tip electrode 164 for use with the tip electrode 164 for bipolar pacing of the right and left ventricles via the His-Purkinje system and/or ventricular myocardium. Ventricular pacing pulses delivered by pacemaker 14 may capture at least a portion of the His bundle and/or ventricular myocardium for delivering ventricular pacing from an atrial implant location of pacemaker 14. The techniques disclosed herein are not necessarily limited to a particular implant location of pacemaker 14, however, and may be practiced in a pacemaker implanted in a variety of operative locations for providing cardiac signal sensing of atrial and/or ventricular events and, at least in some examples, delivering cardiac pacing to at least one heart chamber. For example, pacemaker 14 may be implanted within the right ventricle, e.g., along the interventricular septum just below the tricuspid valve at a location where P-waves and R-waves could be sensed by pacemaker 14. In still other examples, pacemaker 14 may be implanted outside the heart in an epicardial or pericardial location for sensing cardiac signals and delivering pacing pulses.

In the illustrative examples presented herein, pacemaker 14 is implanted in an intracardiac location such that cardiac signals sensed and processed by pacemaker 14 may be referred to as cardiac EGM signals. The techniques disclosed herein may be particularly useful for sensing and discriminating between P-waves and R-waves when pacemaker 14 is implanted within the right atrium in the Triangle of Koch for providing dual chamber sensing and ventricular pacing or dual chamber sensing and dual chamber pacing. For instance, the tip electrode 164 may be advanced toward the interventricular septum into a position in the ventricular myocardium, e.g., in the area of the His bundle, with distal ring electrode 165 making contact or within operative proximity with the atrial myocardium. Proximal ring electrode 162 may thereby be positioned in the blood pool of the right atrium. A ventricular sensing electrode vector between tip electrode 164 and ring electrode 162 for sensing a ventricular EGM signal and an atrial sensing electrode vector between ring electrode 165 and ring electrode 162 for sensing an atrial EGM signal may share a common sensing anode electrode 162. In this arrangement, the proximity of the ventricular sensing cathode, tip electrode 164, to atrial tissue and the proximity of the atrial sensing cathode, ring electrode 165, to ventricular tissue can result in far field P-waves present in the ventricular signal and far field R-waves present in the atrial signal. The techniques disclosed herein provide methods for reliably sensing R-waves and P-waves from the ventricular EGM signal and the atrial EGM signal, respectively, without oversensing of far field signals when pacemaker 14 is implanted in the right atrium, e.g., at a target site in the Triangle of Koch. In some examples, the techniques disclosed herein may be utilized for reliably sensing and discriminating P-wave and/or R-waves, and T-waves if desired, from a single EGM signal sensed between an available sensing electrode vector.

It is to be understood that pacemaker 14 or another medical device such as a cardiac monitor or an implantable cardioverter defibrillator, may be positioned at other locations including outside the heart and be configured to perform aspects of the techniques disclosed herein. A medical device performing cardiac event sensing according to techniques disclosed herein may be coupled to electrodes which may be implanted transvenously within or outside the heart, implanted non-transvenously, e.g., in a subcutaneous, submuscular, substernal or pericardial location for sensing cardiac signals from outside the heart as electrocardiogram (ECG) signals. In some examples, the techniques may be employed in a medical device coupled to external or surface electrodes positioned on the skin of the patient. Electrodes 162, 164 and 165 shown in FIG. 1 are leadless, housing-based electrodes located on the housing of pacemaker 14. However, in other examples, electrodes used for sensing cardiac signals may include one or more lead-based electrodes, carried by a medical electrical lead extending from the medical device to a desired cardiac signal sensing location.

While the techniques disclosed herein are generally described in conjunction with pacemaker 14 capable of dual chamber sensing of cardiac signals and dual chamber cardiac pacing, in other examples, the techniques disclosed herein may be implemented in a cardiac monitor, pacemaker, or implantable cardioverter defibrillation or other cardiac device configured for single chamber, dual chamber or multi-chamber sensing. An IMD performing the sensing techniques disclosed herein may or may not include therapy delivery capabilities such as cardiac pacing and/or cardioversion/defibrillation shock delivery.

Pacemaker 14 may be capable of bidirectional wireless communication with an external device 20 for programming sensing and pacing control parameters. External device 20 can be referred to as a “programmer” because it may be used by a physician, technician, nurse, clinician or other qualified user for programming operating parameters in an implantable medical device. External device 20 may be located in a clinic, hospital or other medical facility. External device 20 may alternatively be embodied as a home monitor or a handheld device that may be used in the patient's home or another location. Operating parameters, including sensing and therapy delivery control parameters, may be programmed into pacemaker 14 by a user interacting with external device 20. Data may be retrieved from pacemaker 14 by external device 20 for facilitating patient monitoring by a clinician.

External device 20 may include a processor 52, memory 53, display unit 54, user interface 56 and telemetry unit 58. Processor 52 controls external device operations and processes data and signals received from pacemaker 14. Display unit 54 may generate a display, which may include a graphical user interface, of data and information relating to pacemaker functions to a user for reviewing pacemaker operation and programmed parameters. Display unit 54 may generate a display that includes cardiac signals and/or data derived therefrom, cardiac pacing timing markers, cardiac pacing history and/or other physiological data, patient data or device-related data that may be stored by pacemaker 14 and transmitted to external device 20 during an interrogation session. For example, pacemaker 14 may generate an output for transmission to external device 20 including pacing and sensing event histories, device operating parameters and device diagnostic data.

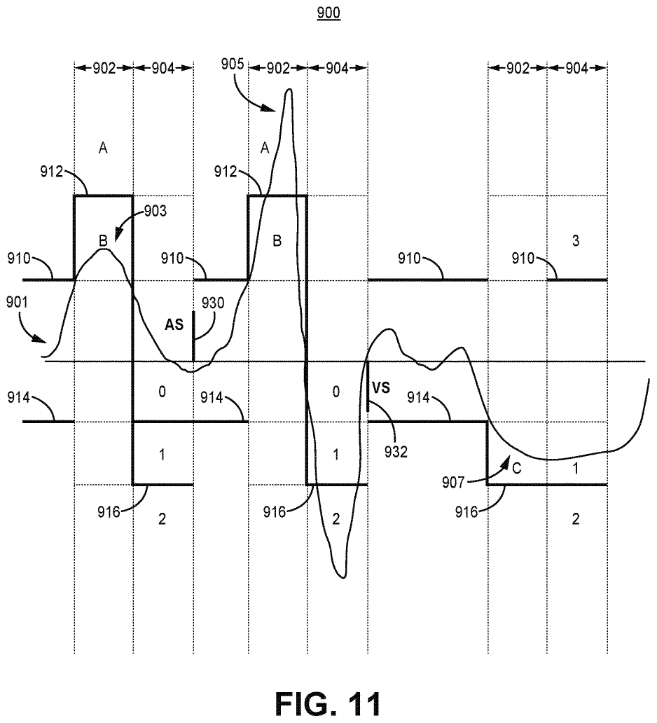

Transmitted data may include an episode of a cardiac electrical signal produced by pacemaker sensing circuitry including markers indicating pacing pulse delivery and sensed cardiac event signals, e.g., ventricular sensed event signals and/or atrial sensed event signals and any delivered atrial and/or ventricular pacing pulses. The display unit 54 may display a cardiac electrical signal episode with annotated sensed event signals and pacing pulse markers, for example. Examples of a cardiac signal and programmable sensing control parameters that may be displayed in a graphical user interface by display unit 54 for facilitating programming of pacemaker 14 are described below, e.g. in conjunction with FIG. 11.

User interface 56 may include a mouse, touch screen, keypad or the like to enable a user to interact with external device 20 to initiate a telemetry session with pacemaker 14 for retrieving data from and/or transmitting data to the pacemaker 14, including programmable parameters for controlling sensing and pacing functions. Telemetry unit 58 includes a transceiver and antenna configured for bidirectional communication with a telemetry circuit included in pacemaker 14 and is configured to operate in conjunction with processor 52 for sending and receiving data relating to pacemaker functions via communication link 24.

Telemetry unit 58 may establish a wireless bidirectional communication link 24 with pacemaker 14. Communication link 24 may be established using a radio frequency (RF) link such as BLUETOOTH®, Wi-Fi, Medical Implant Communication Service (MICS) or other communication bandwidth. In some examples, external device 20 may include a programming head that is placed proximate pacemaker 14 to establish and maintain a communication link 24, and in other examples external device 20 and pacemaker 14 may be configured to communicate using a distance telemetry algorithm and circuitry that does not require the use of a programming head and does not require user intervention to maintain a communication link.

It is contemplated that external device 20 may be in wired or wireless connection to a communications network via a telemetry circuit that includes a transceiver and antenna or via a hardwired communication line for transferring data to a centralized database, server or computer to allow remote management of the patient. Remote patient management systems including a centralized patient database or server may enable a clinician to view data relating to sensing and pacing functions performed by pacemaker 14 and may enable a clinician to remotely program pacemaker 14.

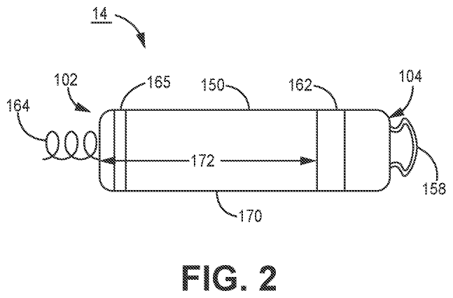

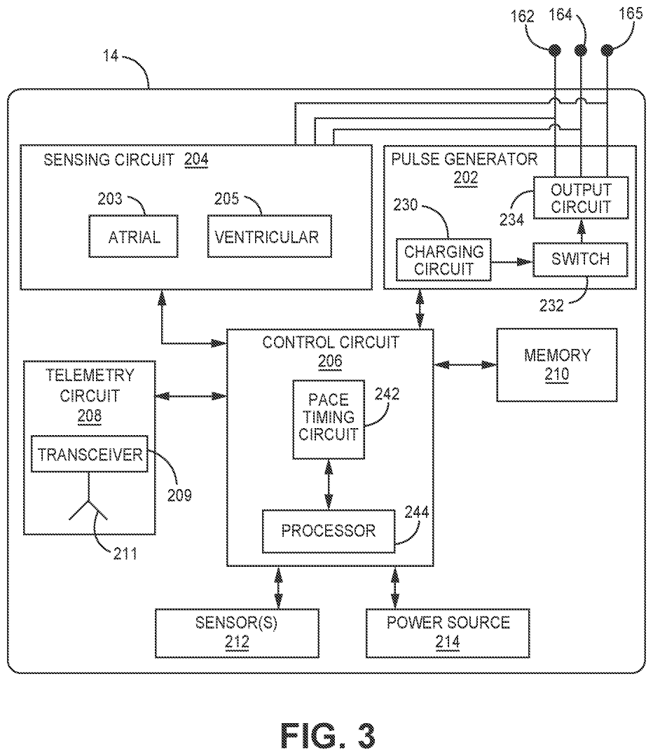

FIG. 2 is a conceptual diagram of the pacemaker 14 shown in FIG. 1 according to one example. Pacemaker 14 includes a housing 150 having a distal end face 102 and a proximal end face 104. The lateral sidewall 170 of housing 150 extending from distal end face 102 to proximal end face 104 may be generally cylindrical to facilitate transvenous delivery, e.g., via a catheter, of pacemaker 14 to an implant site. Distal end face 102 is referred to as “distal” in that it is expected to be the leading end as pacemaker 14 is advanced through a delivery tool, such as a catheter, and placed against a targeted implant site. In other examples, housing 150 may have a generally prismatic shape. The housing 150 encloses the electronics and a power supply for sensing cardiac signals, producing pacing pulses and controlling therapy delivery and other functions of pacemaker 14 as described herein, e.g., in conjunction with FIG. 3.

Pacemaker 14 is shown including electrodes 162, 164 and 165 spaced apart along the housing 150 of pacemaker 14 for sensing cardiac electrical signals and delivering pacing pulses. Electrodes 162, 164 and 165 may be, without limitation, titanium, platinum, iridium or alloys thereof and may include a low polarizing coating, such as titanium nitride, iridium oxide, ruthenium oxide, platinum black, among others. Electrode 164, also referred to herein as “tip electrode” 164, is shown extending from distal end face 102 of housing 150. Tip electrode 164 is shown as a screw-in helical electrode which may provide fixation of pacemaker 14 at an implant site as well as serving as a pacing and sensing electrode. Electrode 164 can be advanced from within the right atrial chamber to a ventricular pacing site, e.g., toward or into the interventricular septum, for delivering pacing to the His-Purkinje conduction system and/or for pacing of ventricular septal myocardial tissue. A proximal portion of tip electrode 164, nearest housing distal end face 102, may be provided with an electrically insulative coating. The more distal portion of tip electrode 164, positioned at a target pacing site, may be uninsulated to function as the electrically conductive portion of tip electrode 164 for pacing pulse delivery and for sensing cardiac electrical signals, e.g., a ventricular EGM signal. Examples of insulating coatings that may be provided on the proximal portion of tip electrode 164 include parylene, urethane, polyether ether ketone (PEEK), or polyimide, among others.

A distal ring electrode 165 is shown as a ring electrode circumscribing the lateral sidewall 170 of housing 150. In other examples, electrode 165 may be a dot, button, ring, hemispherical, segmented or other type of electrode positioned on the distal end face 102 of housing 150 and/or along the lateral sidewall 170, e.g., near distal end face 102. A proximal ring electrode 162 is shown as a ring electrode circumscribing the lateral sidewall 170 of housing 150 spaced proximally from distal ring electrode 165, toward proximal end face 104 of housing 150. In other examples, proximal ring electrode 162 may be a dot, button, ring, hemispherical, segmented or other type of electrode positioned on the proximal end face 104 of housing 150 and/or along the lateral sidewall 170, spaced proximally and/or laterally from electrode 165. Electrodes 162 and 165 may both be ring electrodes circumscribing the lateral sidewall 170 in some examples, e.g., adjacent proximal end face 104 and adjacent distal end face 102, respectively. Other portions of housing 150 may be electrically insulated by an insulating coating.

Tip electrode 164 may serve as a cathode electrode with ring electrode 162 serving as a return anode for delivering ventricular pacing pulses, which may be delivered to capture of at least a portion of the His-Purkinje system and/or ventricular myocardium. Tip electrode 164 and ring electrode 162 may be used as a bipolar pair for ventricular pacing and for receiving a ventricular electrical signal from which R-waves can be sensed by sensing circuitry enclosed by housing 150. Electrodes 165 and 162 may form a second cathode and return anode pair for bipolar atrial pacing and sensing an atrial electrical signal from which P-waves can be sensed by the sensing circuitry enclosed by housing 150. In some examples, any combination of electrodes 162, 164 and 165 may be used in an electrode sensing vector for sensing one or more cardiac electrical signals from which P-waves and/or R-waves may be sensed according to the techniques disclosed herein.

Electrodes 162, 164 and 165 may be positioned at locations along pacemaker 14 other than the locations shown. Examples of various pacing electrode arrangements for providing cardiac pacing along the native conduction system of the heart and/or ventricular myocardium are generally disclosed in U.S. Pat. No. 11,426,578 (Yang, et al.) and U.S. Pat. No. 11,007,369 (Sheldon, et al.), both of which are incorporated herein by reference in their entirety.

In some instances, when tip electrode 164 and ring electrode 162 are used for sensing ventricular R-waves from a RA implant location, atrial P-waves may be oversensed as false R-waves. When ring electrodes 162 and 165 are used for sensing atrial P-waves, far field R-waves may be oversensed as false P-waves. These types of “cross-chamber” oversensing may occur when the electrodes used for sensing a cardiac electrical signal from one heart chamber are in close proximity to another heart chamber, e.g., as in the case of pacemaker 14 positioned in the RA for delivering ventricular pacing as generally shown in FIG. 1, e.g., with tip electrode 164 advanced in the Triangle of Koch. As described herein, pacemaker 14 may be configured to sense both P-waves and R-waves in some examples for controlling ventricular pacing pulse delivery in various pacing modes, which may include both atrial synchronous ventricular pacing modes and asynchronous ventricular pacing modes. Whether pacemaker 14 is configured to sense ventricular R-waves, atrial P-waves or both, pacemaker 14 may be configured to sense cardiac event signals according to the techniques disclosed herein to avoid cross chamber oversensing from interfering with the scheduling and delivery of cardiac pacing pulses according to a pacing mode.

As described below, R-waves may be sensed from a ventricular sensing electrode vector, e.g., between tip electrode 164 and proximal ring electrode 162. P-waves may be sensed from an atrial sensing electrode vector, e.g., between distal ring electrode 165 and proximal ring electrode 162. In still other examples, R-waves and P-waves may be sensed from the signal received via a single sensing electrode vector, e.g., between either of tip electrode 164 or distal ring electrode 165 and the proximal ring electrode 162. The polarity and amplitude of the R-wave and the P-wave in a received cardiac electrical signal in any of the sensing electrode vectors may vary depending on the interelectrode distance 172 between the distal end 102 and the proximal ring electrode 162. Other factors that can influence the polarity and amplitude of the R-waves and P-wave include the relative alignment of pacemaker housing 150 with the cardiac axis. The techniques disclosed herein provide flexibility in selecting P-wave sensing criteria and R-wave sensing criteria applied to a single cardiac electrical signal or two different cardiac electrical signals for reliably sensing and discriminating P-waves and R-waves having varying morphologies due to relative positioning of pacemaker 14 within the heart, anatomical differences, inter-electrode distance, and other factors.

Housing 150 is formed from a biocompatible material, such as a stainless steel or titanium alloy. In some examples, the housing 150 may include an insulating coating. Examples of insulating coatings include parylene, urethane, PEEK, or polyimide, among others. The entirety of the housing 150 may be insulated, but only electrodes 162, 164 and 165 uninsulated. Electrodes 162, 164 and 165 are electrically coupled to internal circuitry, e.g., a pacing pulse generator and cardiac electrical signal sensing circuitry, enclosed by housing 150. Electrodes 162 and 165 may be formed as a conductive portion of housing 150 defining respective electrodes that are electrically isolated from each other and from the other portions of the housing 150 as generally shown in FIG. 2.

Pacemaker 14 may include features for facilitating deployment to and fixation at an implant site. For example, pacemaker 14 may optionally include a delivery tool interface 158. Delivery tool interface 158 may be located at the proximal end 104 of pacemaker 14 and is configured to connect to a delivery device, such as a catheter, guidewire or other tool used to position pacemaker 14 at an implant location during an implantation procedure. The delivery tool interface may enable a clinician to advance, retract and steer pacemaker 14 to an implant site and rotate pacemaker 14 to advance the helical tip electrode 164 into the cardiac tissue. Helical tip electrode 164 in this example provides fixation of pacemaker 14 at the implant site. In other examples, however, pacemaker 14 may include a set of fixation tines or other fixation members to secure pacemaker 14 to cardiac tissue. Numerous types of active and/or passive fixation members may be employed for anchoring or stabilizing pacemaker 14 in an implant position.

FIG. 3 is a conceptual diagram of an example configuration of pacemaker 14 according to some examples. Pacemaker 14 may include a pulse generator 202, a cardiac electrical signal sensing circuit 204, a control circuit 206, telemetry circuit 208, memory 210, sensor(s) 212 and a power source 214. The various circuits represented in FIG. 3 may be combined on one or more integrated circuit boards which include an application specific integrated circuit (ASIC), an electronic circuit, a processor (shared, dedicated, or group) and memory that execute one or more software or firmware programs, a combinational logic circuit, state machine or other suitable components that provide the described functionality.

Cardiac electrical signal sensing circuit 204, referred to hereafter as “sensing circuit” 204, is configured to receive at least one cardiac electrical signal via electrodes coupled to pacemaker 14, e.g., via electrode 164 and electrode 162. When electrode 165 is present as shown in FIG. 2, a second cardiac electrical signal may be received via electrodes 165 and 162 and/or electrodes 164 and 165. As such, sensing circuit 204 may have multiple sensing channels for receiving multiple cardiac electrical signals. In the example shown, sensing circuit 204 includes an atrial sensing channel 203 and a ventricular sensing channel 205, however two separate sensing channels are not necessarily required for implementing techniques disclosed herein.

Atrial sensing channel 203 and ventricular sensing channel 205 may each receive a cardiac electrical signal sensed by two different sensing electrode vectors selected from electrodes 162, 164 and 165. Sensing circuit 204 may include switching circuitry for selectively coupling a sensing electrode pair to a respective sensing channel 203 or 205. In other examples, atrial sensing channel 203 and ventricular sensing channel 205 may each receive a common cardiac electrical signal sensed using a sensing electrode vector selected from electrodes 162, 164 and 165. In this case, different filtering and/or other processing of the received signal may be applied by each of atrial sensing channel 203 and ventricular sensing 205 for sensing atrial event signals and ventricular event signals, respectively.

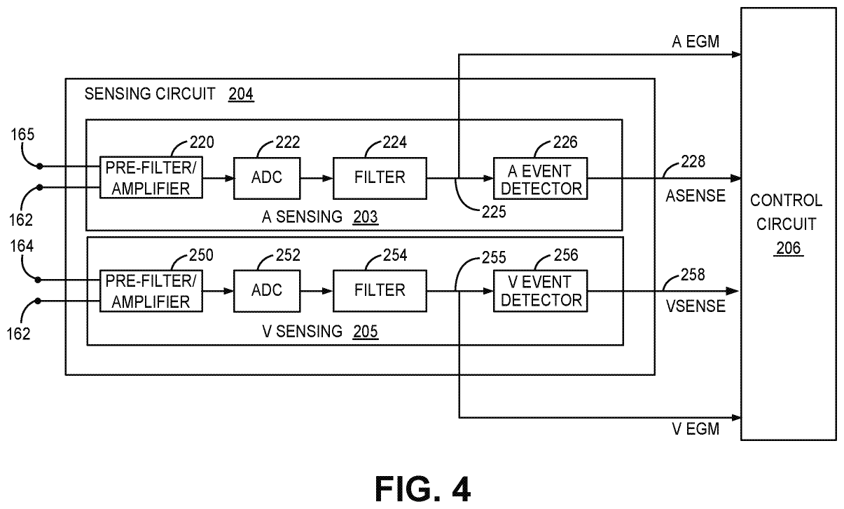

As described below in conjunction with FIG. 4, sensing channels 203 and 205 may include filters, amplifiers, analog-to-digital converters (ADCs), sense amplifiers, comparators, and/or other circuitry for sensing cardiac event signals, e.g., P-waves and/or R-waves, and producing atrial sensed event signals (Asense signals) and ventricular sensed event signals (Vsense signals) that can be passed to control circuit 206. Sensing circuit 204 may be configured to pass a filtered and amplified multi-bit digital cardiac electrogram (EGM) signal to control circuit 206, e.g., from one or both of atrial and ventricular sensing channels 203 and 205. The EGM signal(s) may be processed and analyzed by control circuit 206 for determining a heart rhythm and/or stored in memory 210 as cardiac signal episodes that can be transmitted by telemetry circuit 208, e.g., to external device 20 (shown in FIG. 1). As described below, in some examples the EGM signal(s) may be analyzed for determining if cardiac event sensing criteria are met for confirming a sensed cardiac event signal according to the techniques disclosed herein.

Control circuit 206 may include a pace timing circuit 242 and processor 244. As described below in conjunction with FIG. 4, control circuit 206 may receive Vsense signals and Asense signals from sensing circuit 204 for use in determining cardiac event intervals and/or controlling the timing of cardiac pacing pulses. Vsense signals may be passed from sensing circuit 204 to control circuit 206 in response to ventricular sensing channel 205 sensing a ventricular event signal to indicate the timing of a sensed R-wave. Asense signals may be passed from sensing circuit 204 to control circuit 206 in response to atrial sensing channel 203 sensing an atrial event signal to indicate the timing of a sensed P-wave.

Processor 244 may pass sensing control parameters to sensing circuit 204 for use in sensing cardiac event signals from the cardiac electrical signal(s). For example, programmable or default values of one or more blanking periods, refractory periods, atrial sensitivity, ventricular sensitivity, and other control parameters used by sensing circuit 204 for applying sensing threshold amplitudes and other criteria for sensing cardiac event signals may be passed to sensing circuit 204 from processor 244. Techniques for sensing cardiac event signals and confirming sensed cardiac event signals as being atrial or ventricular event signals are further described below.

Processor 244 may include one or more clocks for generating clock signals that are used by pace timing circuit 242 to time out various pacing intervals for providing atrial and/or ventricular pacing according to an operating pacing mode. Pace timing circuit 242 may start a pacing interval, e.g., by starting an escape interval timer, to schedule a pacing pulse based on the operating pacing mode of control circuit 206. Control circuit 206 may be configured to operate in a variety of programmable and/or automatically switchable pacing modes. When pacemaker 14 is operating in a single chamber atrial pacing mode or a dual chamber pacing mode, pace timing circuit 242 may schedule atrial pacing pulses at atrial lower rate intervals, which at times may be set to a temporary lower rate interval according to a rate smoothing interval or according to a rate response pacing interval.

Pace timing circuit 242 may schedule an atrial pacing pulse by starting an escape interval timer set to the atrial lower rate interval in response to receiving an Asense signal from sensing circuit 204 or in response to pulse generator 202 delivering an atrial pacing pulse. In some examples, as described below, control circuit 206 may receive an Asense signal from sensing circuit 204 and may wait for an Asense confirmation signal or perform analysis of a received atrial EGM signal and/or ventricular EGM signal for confirming the Asense signal. If the atrial pacing interval expires, e.g., when the escape interval timer times out, without receiving an Asense signal (or without confirmation of the Asense signal), pulse generator 202 may deliver an atrial pacing pulse. Pace timing circuit 242 may restart the escape interval timer. If an Asense signal is received (and confirmed) before the atrial pacing interval expires, pace timing circuit 241 may restart the escape interval timer and cancel the scheduled atrial pacing pulse. If an Asense signal is received but not confirmed before the atrial pacing interval expires, pulse generator 202 may be controlled to delay the atrial pacing pulse until the received Asense signal is either confirmed or not confirmed. The delayed, pending atrial pacing pulse may be cancelled when the received Asense signal is subsequently confirmed. The pending atrial pacing pulse may be delivered at a short delay when the received Asense signal is not subsequently confirmed.

During an atrial synchronous ventricular pacing mode, which may be denoted as a DDD or VDD pacing mode as examples, ventricular pacing pulses may be delivered synchronously with atrial pacing pulses and/or received Asense signals. For example, in response to receiving an Asense signal, pace timing circuit 242 may start an AV pacing interval, sometimes referred to as an “AV delay,” to control the timing of an atrial synchronous ventricular pacing pulse. When a ventricular pacing pulse is delivered by pulse generator 202 upon expiration of the AV pacing interval, pace timing circuit 242 may start a ventricular pacing interval (e.g., by starting a pacing escape interval timer) to schedule a ventricular pacing pulse at a programmed ventricular lower rate interval. During atrial synchronous ventricular pacing, if an Asense signal or a Vsense signal is not received prior to the expiration of a ventricular pacing interval, pulse generator 202 may deliver an asynchronous pacing pulse and restart the ventricular pacing interval. The scheduled ventricular pacing pulse can be inhibited if an Asense signal is received (or an atrial pacing pulse is delivered) before the ventricular pacing interval expires. The pending pacing pulse may be cancelled and an atrial synchronous ventricular pacing pulse can be delivered at the AV pacing interval from the Asense signal (or delivered atrial pacing pulse).

In response to receiving a Vsense signal from sensing circuit 204, pace timing circuit 242 may inhibit a pending ventricular pacing pulse scheduled at the ventricular pacing interval (or scheduled at an AV pacing interval) and restart the ventricular pacing interval. The ventricular pacing interval may be a lower rate interval (LRI) corresponding to a programmed minimum or base ventricular pacing rate. In other instances, the ventricular pacing interval may be a temporary ventricular pacing interval set to a rate smoothing interval to avoid an abrupt change in ventricular rate. In other instances, the ventricular pacing interval may be a temporary rate response pacing interval set to provide rate response pacing during increased patient physical activity. If a Vsense signal is received but not confirmed before a ventricular pacing interval expires, pulse generator 202 may be controlled to delay the ventricular pacing pulse until the received Vsense signal is either confirmed or not confirmed. The delayed, pending ventricular pacing pulse may be cancelled when the received Vsense signal is subsequently confirmed. The pending ventricular pacing pulse may be delivered at a short delay when the received Vsense signal is not subsequently confirmed.

Pulse generator 202 generates electrical pacing pulses that can be delivered to pace the ventricles of the patient's heart via cathode electrode 164 and return anode electrode 162. In examples including atrial pacing capabilities, pulse generator 202 may generate electrical pacing pulses for pacing the atria, e.g., using electrodes 165 and 162. In addition to providing control signals to pace timing circuit 242 and pulse generator 202 for controlling the timing of pacing pulses, processor 244 may retrieve programmable pacing control parameters from memory 210, such as pacing pulse amplitude and pacing pulse width, which are passed to pulse generator 202 for controlling pacing pulse delivery.

Pulse generator 202 may include charging circuit 230, switching circuit 232 and an output circuit 234. Charging circuit 230 is configured to receive current from power source 214 and may include a holding capacitor that may be charged to a pacing pulse amplitude, e.g., under the control of a voltage regulator included in charging circuit 230. The pacing pulse amplitude may be set based on a control signal from control circuit 206. Switching circuit 232 may control when the holding capacitor of charging circuit 230 is coupled to the output circuit 234 for delivering the pacing pulse. For example, switching circuit 232 may include a switch that is activated by a timing signal received from pace timing circuit 242 upon expiration of a pacing escape interval and kept closed for a programmed pacing pulse width to enable discharging of the holding capacitor of charging circuit 230. The holding capacitor, previously charged to the pacing pulse voltage amplitude, can be discharged across electrodes 164 and 162 (or 165 and 162) through an output capacitor of output circuit 234 for the programmed pacing pulse duration. Various pacing circuitry configurations may be implemented in pacemaker 14 for charging a pacing capacitor or other charge storage device to a predetermined pacing pulse amplitude under the control of control circuit 206 and delivering a pacing pulse.

It is to be understood that when pacemaker 14 is configured for dual chamber pacing, pulse generator 202 may be configured for delivering both atrial and ventricular pacing pulses under the control of pace timing circuit 242. The atrial pacing pulses are generated by pulse generator 202 according to an atrial pacing pulse amplitude and pulse width. The ventricular pacing pulses are generated by pulse generator 202 according to a ventricular pacing pulse amplitude and pulse width. Pulse generator 202 may include an atrial pacing channel and a ventricular pacing channel that may be controlled separately to deliver atrial pacing pulses upon expiration of atrial pacing intervals and deliver ventricular pacing pulses upon expiration of AV pacing intervals and/or ventricular pacing intervals. The separate atrial pacing channel and ventricular pacing channel may include shared circuitry for generating and delivering pacing pulses. For example, atrial and ventricular pacing channels may include shared output circuitry that is selectively coupled to the appropriate pacing electrode pair via switching circuitry included in output circuit 234.

Pacemaker 14 may include one or more sensors 212 for sensing signals correlated to a physiological condition of the patient. For examples, sensor(s) 212 may include an accelerometer, for sensing patient motion. The accelerometer may include a single-axis or multi-axis accelerometer for producing acceleration signals in one or more dimensions, which can be used for determining a relative level of patient physical activity by control circuit 206 in some examples. Pacemaker 14 may be capable of delivering rate response pacing based on a patient physical activity metric determined by control circuit 206 from an acceleration signal produced by motion sensor 212. Control circuit 206 may receive a rectified acceleration signal from motion sensor 212 and determine a patient physical activity metric from the acceleration signal, e.g., by summing acceleration signal sample point amplitudes over an activity metric time interval. The activity metric may be converted to a target heart rate to meet the patient's metabolic demand. The target heart rate may be converted to a sensor indicated rate (SIR) based on an SIR transfer function that includes a lower rate set point and an activities of daily living (ADL) range and a maximum upper rate, for example. During a rate response pacing mode, pulse generator 202 may be controlled by control circuit 206 to deliver atrial or ventricular pacing pulses at a rate response pacing rate determined based on the SIR. In other examples, sensor(s) 212 may include a pressure sensor, heart sound sensor, oxygen sensor, temperature sensor or other sensors used for monitoring a physiological condition of the patient.

Memory 210 may include computer-readable instructions that, when executed by control circuit 206, cause control circuit 206 to perform various functions attributed throughout this disclosure to pacemaker 14. The computer-readable instructions may be encoded within memory 210. Memory 210 may include any non-transitory, computer-readable storage media including any volatile, non-volatile, magnetic, optical, or electrical media, such as a random access memory (RAM), read-only memory (ROM), non-volatile RAM (NVRAM), electrically-erasable programmable ROM (EEPROM), flash memory, or other digital media with the sole exception being a transitory propagating signal.

Telemetry circuit 208 includes a transceiver 209 and antenna 211 for transferring and receiving data via a radio frequency (RF) communication link. Telemetry circuit 208 may be capable of bi-directional communication with external device 20 (FIG. 1) as described above. Cardiac electrical signals and/or data derived therefrom such as sensed event data may be transmitted by telemetry circuit 208 to external device 20. Programmable control parameters and algorithms for sensing cardiac event signals and controlling cardiac pacing therapies delivered by pulse generator 202 may be received by telemetry circuit 208 and stored in memory 210 for access by control circuit 206.

Power source 214 provides power to each of the other circuits and components of pacemaker 14 as required. Power source 214 may include one or more energy storage devices, such as one or more rechargeable or non-rechargeable batteries. The connections between power source 214 and other pacemaker circuits and components are not shown in FIG. 3 for the sake of clarity but are to be understood from the general block diagram of FIG. 3. Power source 214 may provide power as needed to pulse generator 202, sensing circuit 204, telemetry circuit 208, memory 210 and sensor(s) 212.

The functions attributed to pacemaker 14 herein may be embodied as one or more processors, controllers, hardware, firmware, software, or any combination thereof. Depiction of different features as specific circuitry is intended to highlight different functional aspects and does not necessarily imply that such functions must be realized by separate hardware, firmware or software components or by any particular circuit architecture. Rather, functionality associated with one or more circuits described herein may be performed by separate hardware, firmware or software components, or integrated within common hardware, firmware or software components. For example, the term “sensing circuitry” as used herein may refer to circuits and components represented by sensing circuit 204, control circuit 206 or a combination of both configured to perform the cardiac event signal sensing according to the techniques disclosed herein. The term “control circuitry” as used herein may refer to circuits and components represented by sensing circuit 204, control circuit 206 or a combination of both to perform a response to a sensed cardiac event signal such as starting one or more timing control intervals as described below. Providing software, hardware, and/or firmware to accomplish the described functionality in the context of any modern pacemaker, given the disclosure herein, is within the abilities of one of skill in the art.

FIG. 4 is a conceptual diagram of sensing circuitry of pacemaker 14 according to some examples. Sensing circuit 204 may include atrial sensing channel 203 and ventricular sensing channel 205. Atrial sensing channel 203 is shown to receive a raw cardiac electrical signal from electrodes 165 and 162. Ventricular sensing channel 205 is shown to receive a raw cardiac electrical signal from electrodes 164 and 162. However, it is to be understood that other sensing electrode vectors may be provided as input to sensing channels 203 and/or 205 depending on what electrodes are included on pacemaker housing 150 (or coupled to pacemaker 14 via a lead) and which sensing electrode vectors provide the greatest sensitivity for sensing P-waves and R-waves with the greatest discrimination between true P-waves and true R-waves. For instance, ventricular sensing channel 205 may receive a raw cardiac electrical signal from electrodes 164 and 165 in some examples.

The raw cardiac electrical signals are received as input to a pre-filter and amplifier circuit 220 or 250 of atrial sensing channel 203 or ventricular sensing channel 205, respectively. Pre-filter and amplifier circuits 220 and 250 may each include a high pass filter to remove DC offset, e.g., a 2.5 to 5 Hz high pass filter, or a wideband filter having a bandpass of 2.5 Hz to 100 Hz or narrower to remove DC offset and high frequency noise. Pre-filter and amplifier circuits 220 and 250 may further include an amplifier to amplify the raw cardiac electrical signal passed to a respective ADC 222 or 252.

ADC 222 may pass a digital signal to filter 224 of atrial sensing channel 203. Filter 224 may be a bandpass filter having bandpass cutoff frequencies for passing P-wave signals and attenuating other cardiac event signals, e.g., R-waves and T-waves. In some examples, the high pass and/or low pass cutoff frequencies of filter 224 are user programmable and/or may be adjusted by control circuit 206 to attenuate far-field R-waves by atrial sensing channel 203 and/or improve the discrimination between true P-waves and far field R-waves. The low pass cutoff frequency of filter 224 may be between 25 Hz and 100 Hz or between 50 and 100 Hz and the high pass cutoff frequency may be between 2.5 Hz and 25 Hz or between 2.5 and 20 Hz, as examples. The output of filter 224 may be referred to as an atrial electrogram (EGM) signal 225 that can be passed to control circuit 206 and to atrial event detector circuit 226. Filter 224 may be provided as a non-ringing filter to minimize or eliminate ringing artifact and maximally preserve the raw P-wave morphology in the cardiac electrical signal passed to control circuit 206 or atrial event detector circuit 226. The atrial EGM signal passed to atrial event detector circuit 226 can be a non-rectified signal to enable comparison of the signal to a positive atrial sensing threshold amplitude and a negative atrial sensing threshold amplitude in some examples. In other examples, atrial event detector circuit 226 may apply one atrial sensing threshold, having either a positive amplitude or a negative amplitude, to the incoming non-rectified atrial EGM signal.

Atrial event detector circuit 226 may include sense amplifiers, comparators, timers and/or other event detection circuitry that can compare the incoming, filtered and amplified atrial EGM signal to at least one atrial sensing threshold. For example, when the incoming non-rectified signal crosses a first atrial sensing threshold, the atrial event detector circuit 226 may produce an atrial sensed event signal (Asense) 228 that can be passed to control circuit 206. As further described below, in some examples atrial event detector circuit 226 may start one or more confirmation windows (e.g., confirmation time interval(s)) in response to the first atrial sensing threshold crossing and apply a second atrial sensing threshold to the incoming atrial EGM signal for confirming the Asense signal. If an atrial pacing interval expires before an Asense signal 228 is received by control circuit 206, pulse generator 202 (shown in FIG. 3) may deliver an atrial pacing pulse.

The atrial sensing threshold(s) (which could also be referred to as P-wave sensing threshold(s)”) can be fixed or auto-adjusting threshold(s) that is/are automatically decreased in absolute amplitude by sensing circuit 204 from a starting value to a minimum value or until an atrial sensing threshold crossing occurs. For example, a first atrial sensing threshold may initially be set to a starting value (e.g., in millivolts) applied to the atrial EGM signal upon expiration of an atrial blanking period and can be adjusted toward a minimum sensing threshold or “sensing floor” that may be equal to a programmed atrial sensitivity.

Atrial event detector circuit 226 may include a peak track and hold circuit or other circuitry for detecting the maximum peak amplitude of the atrial EGM signal during a portion of the post-atrial blanking period and/or during a cardiac sensed event confirmation window. In some examples, an atrial sensing threshold starting value may be set based on the maximum peak amplitude, e.g., to a percentage of the maximum peak amplitude following an atrial sensing threshold crossing. In some examples, the atrial sensing threshold may be set to 50% to 80% of the maximum peak amplitude. The atrial sensing threshold may be decreased according to one or more decay rates and corresponding decay time intervals and/or one or more stepwise decrements until the atrial EGM signal crosses the atrial sensing threshold or the atrial sensitivity is reached. The atrial sensitivity defines the minimum atrial EGM signal amplitude that can be sensed as a P-wave. In other examples, a first atrial sensing threshold may be set to a fixed amplitude having a first polarity and a second atrial sensing threshold may be set to a starting amplitude having a second polarity opposite the first polarity and be auto-adjusted toward a programmed atrial sensitivity.

ADC 252 of ventricular sensing channel 205 may pass a digitized signal to filter 254. Filter 254 may be a non-ringing, bandpass filter having bandpass cutoff frequencies for passing R-wave signals with a maximally preserved morphology of the raw R-wave and minimized ringing artifact and in some cases attenuation of other cardiac event signals, e.g., P-waves and T-waves. A ventricular EGM signal 255 output by filter 254 may be passed to control circuit 206. The low pass cutoff frequency of filter 254 may be between 25 Hz and 100 Hz or between 50 and 100 Hz and the high pass cutoff frequency may be between 2.5 Hz and 25 Hz or between 2.5 and 20 Hz, as examples. The bandpass cutoff frequencies of filter 254 may be the same or different than the bandpass cutoff frequencies of filter 224.

Ventricular event detector circuit 256 may include one or more sense amplifiers, comparators, timers, peak detectors, and/or other event detection circuitry that compares the incoming, filtered and amplified ventricular EGM signal to at least one ventricular sensing threshold. For example, when the incoming signal crosses a first ventricular sensing threshold having a defined amplitude and polarity, the ventricular event detector circuit 256 may produce a ventricular sensed event signal (Vsense) 258 that can be passed to control circuit 206. As further described below, in some examples ventricular event detector circuit 256 may start one or more confirmation windows (e.g., one or more confirmation time interval(s)) in response to the first ventricular sensing threshold crossing and apply a second ventricular sensing threshold, which may have the opposite polarity as the first ventricular sensing threshold, to the incoming ventricular EGM signal for confirming the Vsense signal. The first ventricular sensing threshold and/or the second ventricular sensing threshold (which could be referred to as “R-wave sensing thresholds”) may be a fixed amplitude or an auto-adjusting threshold amplitude that is automatically decreased by sensing circuit 204. For instance, the first ventricular sensing threshold amplitude may be set to a starting value applied to the ventricular EGM signal upon expiration of a post-ventricular blanking period and can be adjusted toward a minimum ventricular sensing threshold or “sensing floor” that may be equal to a programmed ventricular sensitivity. Additionally or alternatively, the second ventricular sensing threshold may be set to a starting amplitude (of opposite polarity as the first ventricular sensing threshold) upon the first threshold crossing and may be adjusted toward the programmed ventricular sensitivity.

Ventricular event detector circuit 256 may include a peak track and hold circuit or other circuitry for detecting the maximum peak amplitude of the ventricular EGM signal following a ventricular sensing threshold crossing during a peak tracking portion of the post-ventricular blanking period and/or during a cardiac sensed event confirmation window. In some examples, the first (and/or second) ventricular sensing threshold starting value may be set based on the maximum peak amplitude, e.g., to a percentage of the maximum peak amplitude. In some examples, the ventricular sensing threshold may be set to 50 to 80% of the maximum peak amplitude. The ventricular sensing threshold may be decreased according to one or more decay rates and corresponding decay time intervals and/or one or more stepwise decrements until the ventricular EGM signal crosses the ventricular sensing threshold or the ventricular sensitivity is reached. The ventricular sensitivity may define the minimum ventricular EGM signal amplitude that can be sensed as an R-wave. If a ventricular pacing interval or an AV pacing interval expires before a Vsense signal 258 is received by control circuit 206, pulse generator 202 (shown in FIG. 3) may deliver a ventricular pacing pulse.

Control circuit 206 may provide sensing control signals to sensing circuit 204. Sensing control parameters may include ventricular sensing threshold adjustment parameters, e.g., the percentage of the maximum peak amplitude used for setting the starting sensing threshold amplitude(s) and the ventricular sensitivity. Control circuit 206 may provide sensing control signals to sensing circuit 204 used for atrial sensing threshold adjustment parameters, e.g., the percentage of the maximum peak amplitude used for setting the starting atrial sensing threshold(s) and the atrial sensitivity. Sensing control parameters may include various timing control intervals such as blanking and refractory intervals applied to the atrial EGM signal, e.g., a post-sense atrial blanking period, a post-pace atrial blanking period, an atrial refractory period and a post-ventricular atrial blanking period. Sensing control parameters may include various blanking and refractory intervals applied to the ventricular EGM signal, e.g., a post-sense ventricular blanking period, a post-pace ventricular blanking period, a ventricular refractory period, and a post-atrial ventricular blanking period.

As described below, one or both of atrial event detector circuit 226 and ventricular event detector circuit 256 may apply both a positive sensing threshold amplitude and a negative sensing threshold amplitude to a respective incoming, non-rectified atrial EGM signal or ventricular EGM signal (or common EGM signal). When the electrodes included in the atrial sensing electrode vector and the electrodes included in the ventricular sensing electrode vector (or in a common atrial and ventricular sensing electrode vector) are proximate to tissue of both atrial and ventricular heart chambers, the P-waves and R-waves may be difficult to sense discriminately based on a respective single atrial event or ventricular sensing threshold applied to a rectified EGM signal. Cross-chamber oversensing may occur when far field P-waves cross the ventricular sensing threshold or far field R-waves cross the atrial sensing threshold. As such, by retaining polarity information in the non-rectified atrial EGM signal and/or non-rectified ventricular EGM signal (or a single non-rectified EGM signal) and applying both a positive and a negative sensing threshold amplitude, P-waves and R-waves may be sensed discriminately with cross-chamber oversensing avoided or minimized and T-wave oversensing avoided or minimized.

In some examples, the atrial sensing channel 203 and the ventricular sensing channel 205 may function cooperatively for sensing P-waves and R-waves. For example atrial event detector circuit 226 and ventricular event detector circuit 256 may be in communication with each other for enabling one detector circuit 226 or 256 to receive information about the status of the other detector circuit 256 or 226 and/or data determined from the respective atrial EGM signal or ventricular EGM signal. In this way, one detector circuit 226 or 256 may be enabled to confirm a respective atrial sensing threshold crossing or ventricular sensing threshold crossing as a near field sensed cardiac event (e.g., P-wave or R-wave, respectively) as opposed to being an oversensed cross-chamber event (e.g., a far field R-wave or far field P-wave, respectively).

For example, atrial event detector circuit 226 may pass signals to ventricular event detector circuit 256 to facilitate confirmation of a ventricular sensing threshold crossing as being an R-wave by ventricular event detector circuit 256. Examples of signals received by ventricular event detector circuit 256 from atrial event detector circuit 256 may indicate: the timing of an atrial sensing threshold crossing; when an Asense confirmation window is started, running and/or terminated; one or more features of the atrial EGM signal (such as a maximum peak amplitude, slope or other example features listed below); and/or when an atrial sensing threshold crossing is confirmed by atrial event detector circuit 226 as being a sensed atrial event, e.g., a sensed P-wave. Ventricular event detector circuit 256 may use information received from atrial event detector circuit 256 in confirming a ventricular sensing threshold crossing as being an R-wave and or rejecting the ventricular sensing threshold crossing as a likely far field P-wave.

In an analogous manner, ventricular event detector circuit 256 may pass signals to atrial event detector circuit 226 indicating, for example: the timing of a ventricular sensing threshold crossing; when a Vsense confirmation window is started, running or and/or terminated; one or more features of the ventricular EGM signal; and/or when a ventricular sensing threshold crossing is confirmed by ventricular event detector circuit 256 as being a sensed ventricular event, e.g., a sensed R-wave. Atrial event detector circuit 226 may use this information in confirming that an atrial sensing threshold crossing is a sensed P-wave and not a far field R-wave.

In other examples, control circuit 206 may receive the atrial EGM signal 225 and/or the ventricular EGM signal 255 from sensing circuit 204 for confirming or rejecting Asense signals 228 received from atrial sensing channel 203 and/or for confirming or rejecting Vsense signals 258 received from ventricular sensing channel 205. As further described below, cardiac event sensing criteria may be applied to the atrial EGM signal 225 and/or the ventricular EGM signal 255 for confirming Asense signals and/or Vsense signals received by control circuit 206 from sensing circuit 204.

Asense signals 228 and Vsense signals 258 passed from sensing circuit 204 to control circuit 206 may be used for controlling the timing of atrial and/or ventricular pacing pulses by pace timing circuit 242. Pace timing circuit 242 (shown in FIG. 3) may start one or more pacing escape interval timers upon receiving an Asense or Vsense signal. The value reached by an escape interval timer between two consecutive Asense signals or between an Asense signal and a preceding atrial pacing pulse can be determined as an atrial event interval, or PP interval (PPI), for use in determining an atrial rate. The value reached by an escape interval timer between two consecutive Vsense signals or between a Vsense signal and a preceding ventricular pacing pulse can be determined as a ventricular event interval, or RR interval (RRI), for use in determining a ventricular rate. The atrial event intervals or an atrial rate determined therefrom and/or the ventricular event intervals or the ventricular rate determined therefrom may be determined by control circuit 206 for storing cardiac data in memory 210, controlling pacing mode switching, detecting arrhythmias or other pacemaker functions.

When sensing circuit 204 is configured to receive a raw atrial electrical signal and a raw ventricular electrical signal as shown in FIG. 4, components included in an atrial sensing channel 203 and in a ventricular sensing channel 205 may be separate or shared between both sensing channels 203 and 205 in various examples. For example, pre-filter/amplifiers 220 and 250 and/or ADCs 222 and 252 may be shared by both atrial sensing channel 203 and ventricular sensing channel 205 with separate outputs being passed to atrial event detector circuit 226, e.g., via filter 224, and to ventricular event detector circuit 256, e.g., via filter 254. In some cases, different filtering and amplification may be applied to the output of an ADC before passing separate signals to the respective atrial event detector circuit 226 and ventricular event detector circuit 256. For example filter 224 can be tuned to enhance P-wave signal amplitude and attenuate other cardiac signals. Filter 254 can be tuned to enhance R-wave signal amplitude and attenuate other cardiac signals. In other examples, the non-ringing bandpass filtering may be applied to the output of an ADC that is passed to both the atrial event detector circuit 226 and the ventricular event detector circuit 256 which each apply respective sensing thresholds for sensing P-waves and R-waves, respectively.

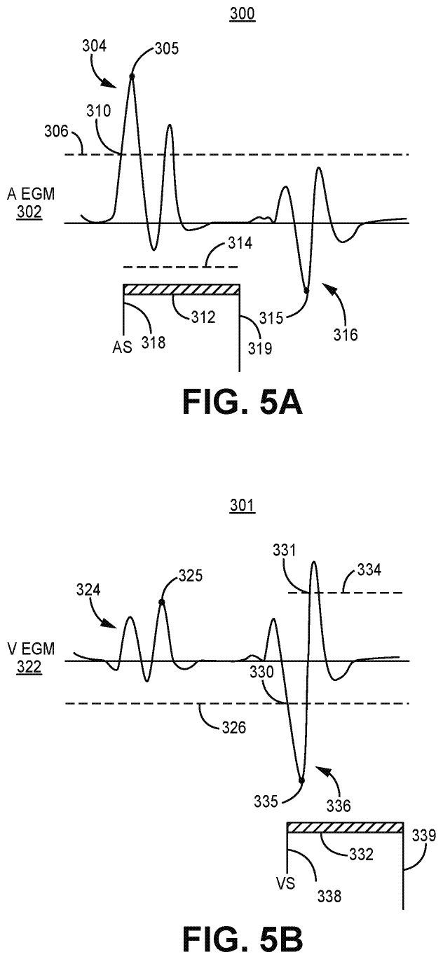

Furthermore, it is to be understood that other sensing electrode vectors than the vectors shown in FIG. 4 may be used for sensing and discriminating between P-waves and R-waves according to the techniques disclosed herein. In the example of pacemaker 14 including tip electrode 164 and two ring electrodes 162 and 165, any combination of the sensing electrode vectors, e.g., between electrodes 164 and 162, electrodes 164 and 165, and/or electrodes 162 and 165 may be received as input to sensing circuit 204 for use in sensing and discriminating between P-waves and R-waves according to the techniques disclosed herein. In still other examples, sensing circuit 204 may receive a cardiac electrical signal from a single sensing electrode vector that is amplified, filtered and passed to a cardiac event detector circuit of sensing circuit 204 and/or control circuit 206 without rectification for comparison to multiple sensing threshold amplitudes including at least one positive sensing threshold amplitude and at least one negative sensing threshold amplitude for identifying P-waves and R-waves from the cardiac electrical signal as further described below in conjunction with FIGS. 5A-12.

FIG. 5A is a diagram 300 of an example atrial EGM signal 302, and FIG. 5B is a diagram 301 of an example ventricular EGM signal 322 that may be received by cardiac event detection circuitry, e.g., included in sensing circuit 204, for sensing P-waves and R-waves, respectively. The atrial EGM signal 302 in FIG. 5A may be digitized and filtered and passed to atrial event detector circuit 226 as a non-rectified signal for sensing P-waves, e.g., P-wave 304. The atrial EGM signal 302 is shown including a P-wave 304 attendant to an atrial depolarization and a far field R-wave 316 attendant to a ventricular depolarization. The ventricular EGM signal 322 in FIG. 5B may be digitized and filtered and passed to ventricular event detector circuit 256 as a non-rectified signal for sensing R-waves, e.g., R-wave 336. The ventricular EGM signal 322 is shown including a far field P-wave 324, corresponding to P-wave 304, and an R-wave 336, corresponding to far field R-wave 316.

Techniques disclosed herein may be implemented in sensing circuit 204 to promote reliable sensing of P-wave 304 from the atrial EGM signal 302 without oversensing of far field R-wave 316 by the atrial event detector circuit 226 of atrial sensing channel 203 (shown in FIG. 4). Additionally or alternatively, the techniques disclosed herein may be implemented by sensing circuit 204 to promote reliable sensing of R-wave 336 from ventricular EGM 322 without oversensing of the far field P-wave 324 by the ventricular event detector circuit 256 of ventricular sensing channel 205 (shown in FIG. 4).

As shown in FIG. 5A, sensing circuit 204 may apply a first atrial sensing threshold 306 having a positive polarity amplitude to the atrial EGM signal 302. In the example shown, the first atrial sensing threshold 306 has a positive polarity, but the first atrial sensing threshold 306 could have a negative polarity in other examples. The polarity of the first atrial sensing threshold 306 applied to the atrial EGM signal 302 may depend on the locations of the electrodes in an atrial sensing electrode vector used to sense the atrial EGM signal and the relative direction of conduction of atrial depolarizations.

In response to detecting a threshold crossing 310 of the first atrial sensing threshold 306 by atrial EGM signal 302, the sensing circuit 204 may start an Asense confirmation window 312. The Asense confirmation window 312 is a time interval that may extend from the time of threshold crossing 310 (or a specified time interval prior to the threshold crossing 310) to a specified time interval after the threshold crossing 310. The Asense confirmation window 312 may be 10 to 80 ms in duration and can be 20 to 50 ms in duration in some examples. The Asense confirmation window 312 may be 30 ms in an example. Sensing circuit 204 may apply other P-wave sensing criteria to the atrial EGM signal 302 and/or the ventricular EGM signal 324 during the Asense confirmation window 312 to confirm the likelihood of the first atrial sensing threshold crossing 310 being a P-wave before generating an Asense signal 318 that is passed to control circuit 2-6. In other examples, the Asense signal 318 may be passed to control circuit 206 upon detection of the threshold crossing 310. However, if P-wave sensing criteria are not confirmed based on the atrial EGM signal 302 and/or ventricular EGM signal 324 sensed by the expiration of the Asense confirmation window 312, control circuit 206 may ignore the Asense signal 318 in controlling pacing and timing operations. In still other examples, sensing circuit 204 may generate an Asense confirmation signal 319 at the expiration of the Asense confirmation window 312 when P-wave sensing criteria are met by the atrial EGM signal 302 and/or the ventricular EGM signal 322 sensed during the Asense confirmation window 312 to confirm the Asense signal 318 as being a P-wave.

For instance, sensing circuit 204 may verify that the ventricular EGM signal 322 does not cross a ventricular sensing threshold amplitude during the Asense confirmation window 312. If the ventricular EGM signal 322 crosses a ventricular sensing threshold amplitude during the Asense confirmation window 312, or a Vsense confirmation window is currently running due to a preceding ventricular sensing threshold crossing, the atrial threshold crossing 310 of the first atrial sensing threshold 306 could be due to a far field R-wave in the atrial EGM signal 302. In this case, sensing circuit 204 may not confirm the threshold crossing 310 as being an atrial sensed event signal because of the possibility of an oversensed far field R-wave.

Additionally or alternatively, sensing circuit 204 may verify that the atrial EGM signal 302 meets a second atrial sensing threshold requirement during the Asense confirmation window 312. A second atrial sensing threshold 314 may be applied to the atrial EGM signal 302 during the Asense confirmation window 312. The second atrial sensing threshold 314 may have the opposite polarity of the first atrial sensing threshold 306. In this illustrative example, the second atrial sensing threshold 314 is a negative polarity amplitude. However, in other examples, the first atrial sensing threshold 306 could be a negative polarity amplitude and the second atrial sensing threshold 314 could be a positive polarity amplitude. The amplitude of the first and second atrial sensing thresholds 306 and 314 and their respective polarities may be programmable by a user and may be in the range of 0.075 to 12 millivolts as examples, with no limitation intended. In an example, the programmable atrial sensing thresholds 306 and 314 may be between 0.15 and 11.3 mV. The absolute values of the amplitudes of the positive and negative first and second atrial sensing threshold amplitudes 306 and 314 may be the same or different from each other and may be individually programmable.

In the example shown, sensing circuit 204 determines that the atrial EGM signal 302 meets the second atrial sensing threshold requirement when the atrial EGM signal does not cross the second atrial sensing threshold 314 during the Asense confirmation window 312. In this case, the atrial sensing electrode vector may be positioned such that a substantially monophasic P-wave 304 is expected with a biphasic far field R-wave 316 in the atrial EGM signal. In other examples, a crossing of the second atrial sensing threshold 314 during the Asense confirmation window 312 may be required for the sensing circuit 204 to determine that the second atrial sensing threshold requirement is met. In this case, the atrial sensing electrode vector may be positioned such that a biphasic P-wave, having an initially positive going waveform, is expected in the atrial EGM signal 302.

When the second atrial sensing threshold requirement is met by the expiration of the Asense confirmation window 312, sensing circuit 204 may generate the Asense signal 318. The Asense signal 318 may be passed to control circuit 206, optionally including a time stamp corresponding to the time of the first atrial sensing threshold crossing 310 (as shown). In other examples, the Asense signal 318 may be generated upon expiration of the Asense confirmation window 312. Control circuit 206 may optionally adjust the time of the atrial sensed event by the time duration of the Asense confirmation window 312. For example, the Asense confirmation window 312 may be subtracted from the time expired on an escape interval timer included in pace timing circuit 242 (FIG. 3) since a most recent preceding Asense signal or delivered atrial pacing pulse for determining an atrial event interval and a corresponding atrial rate. In still other examples, sensing circuit 204 may generate the Asense signal 318 at the time of the first atrial sensing threshold crossing 310 (e.g., as a pending Asense signal) and subsequently generate the Asense confirmation signal 319 when P-wave sensing criteria are met. Control circuit 206 may ignore or cancel the Asense signal 318 if an Asense confirmation signal 319 is not received following the expiration of the Asense confirmation window 312. A time stamp of a pending Asense signal 318 that is subsequently confirmed and/or the Asense confirmation signal 319 may be stored in memory 210 for use by control circuit 206 in determining atrial event intervals (e.g., PPIs) and corresponding atrial rate.

Control circuit 206 and/or sensing circuit 204 may respond to the Asense confirmation signal 319 by using the timing of the received pending Asense signal 318 for starting various timing control intervals and/or determining atrial event intervals. Timing control intervals started in response to a confirmed Asense signal 318 may include an atrial blanking period, an atrial refractory period, a post-atrial ventricular blanking period, an atrial pacing interval, and/or an atrioventricular pacing interval, or an atrial event interval timer as examples. One or more timing control intervals may be started by control circuitry of pacemaker 14 in response to receiving the Asense confirmation signal 319. The timing control interval(s) may be adjusted to have an effective starting time corresponding to the time of threshold crossing 310.

If the Asense signal 318 is not confirmed at the expiration of the Asense confirmation window 312, any currently running timing control intervals may continue running. The Asense signal 318 may be ignored for the purposes of resetting post-atrial sense timing control intervals. If an atrial pacing escape interval expires during the Asense confirmation window 312, a pending atrial pacing pulse may be delayed until the expiration of the Asense confirmation window 312. If the Asense signal 318 is confirmed, the pending atrial pacing pulse may be cancelled. If the Asense signal 318 is not confirmed, the pending atrial pacing pulse may be delivered at the expiration of the Asense confirmation window 312.

In other examples, sensing circuitry of pacemaker 14, which may include components of sensing circuit 204 and/or control circuit 206, may determine the absolute value of the maximum peak amplitude 325 of the ventricular EGM signal 322 (FIG. 5B) during the Asense confirmation window 312 and the absolute value of the maximum peak amplitude 305 of the atrial EGM signal during the Asense confirmation window 312. The pacemaker sensing circuitry may verify that the atrial EGM maximum peak amplitude 305 is greater than ventricular EGM maximum peak amplitude 325 to confirm the likelihood of threshold crossing 310 being associated with a P-wave 304 and not a far field R-wave. If threshold crossing 310 is caused by a far field R-wave, the R-wave in the ventricular EGM signal 322 is expected to have a greater maximum peak amplitude during the Asense confirmation window 312 than the maximum peak amplitude of the atrial EGM signal 302.

Comparison of the absolute maximum peak amplitudes 305 and 325 is one example of a comparison that can be made by sensing circuit 204 (or control circuit 206) between atrial EGM signal 302 and ventricular EGM signal 322 for verifying that the first atrial sensing threshold crossing 310 is likely a P-wave. In other examples, a maximum positive slope, maximum negative slope, minimum peak amplitude, maximum peak amplitude, peak-to-peak amplitude, signal width, number of zero crossings, number of peaks (positive and/or negative), signal area, or other feature(s) determined from atrial EGM signal 302 and ventricular EGM signal 322 sensed during the Asense confirmation window 312 may be determined and compared by sensing circuit 204 and/or control circuit 206 for verifying that P-wave sensing criteria are met.

As shown in FIG. 5B, sensing circuit 204 may apply a first ventricular sensing threshold 326 to the ventricular EGM signal 322. In this example, the first ventricular sensing threshold 326 has a negative polarity amplitude. In other examples, the first ventricular sensing threshold 326 could have a positive polarity amplitude. The polarity of the first ventricular sensing threshold 326 applied to the ventricular EGM signal 302 may be programmable and may be selected based on the locations of the electrodes in the ventricular sensing electrode vector used to sense the ventricular EGM signal 322 and the direction of conduction of ventricular depolarizations relative to the ventricular sensing electrode vector.

In response to detecting a threshold crossing 330 of the first ventricular sensing threshold 326 by ventricular EGM signal 302, the sensing circuit 204 may start a Vsense confirmation window 332. The Vsense confirmation window 332 can be a time window that extends from the threshold crossing 330 or a specified time interval prior to the threshold crossing 330 to a specified time interval after the threshold crossing 310. The Vsense confirmation window 332 may be 10 to 120 ms in duration and can be 20 to 50 ms in duration in some examples, with no limitation intended.

Sensing circuit 204 may apply other R-wave sensing criteria to the ventricular EGM signal 322 and/or the atrial EGM signal 302 sensed during the Vsense confirmation window 332 to confirm the likelihood of the first ventricular sensing threshold crossing 330 being an R-wave that crosses the first ventricular sensing threshold 326 before generating a Vsense signal 338 that is passed to control circuit 206. In other examples, the Vsense signal 338 may be passed to control circuit 206, e.g., as a pending Vsense signal, upon detection of the threshold crossing 330. However, if R-wave sensing criteria are not confirmed based on the atrial EGM signal 302 and/or the ventricular EGM signal 322 sensed up to the expiration of the Vsense confirmation window 332, control circuit 206 may ignore the Vsense signal 338 in controlling pacing and timing operations.

For instance, sensing circuit 204 may determine that R-wave sensing criteria are met by determining that the atrial EGM signal 302 does not cross an atrial sensing threshold amplitude during the Vsense confirmation window 332. If the atrial EGM signal 302 crosses an atrial sensing threshold amplitude during the Vsense confirmation window 332, the first ventricular sensing threshold 306 could be due to a far field P-wave in the ventricular EGM signal 322. If an Asense confirmation window is running during any portion of the Vsense confirmation window 332, e.g., started during or before the Vsense confirmation window 332, due to an atrial sensing threshold crossing, the threshold crossing 330 of the first ventricular sensing threshold 306 could be due to a far field P-wave in the ventricular EGM signal 322. In any of these cases, sensing circuit 204 or control circuit 206 may not confirm the threshold crossing 330 as being a ventricular sensed event signal because of the possibility of an oversensed far field P-wave.

Additionally or alternatively, sensing circuit 204 may verify that the ventricular EGM signal 322 meets a second ventricular sensing threshold requirement during the Vsense confirmation window 332. A second ventricular sensing threshold 334 may be applied to the ventricular EGM signal 322 during the Vsense confirmation window 332. The second ventricular sensing threshold 334 may have the opposite polarity of the first ventricular sensing threshold 326. In the illustrative example of FIG. 5B, the second ventricular sensing threshold 334 is a positive polarity amplitude. However, in other examples, the first ventricular sensing threshold 326 could be a positive polarity amplitude and the second ventricular sensing threshold 334 could be a negative polarity amplitude. The amplitudes of the first and second ventricular sensing thresholds 326 and 334 and their respective polarities may be programmable by a user and may be in the range of 0.075 to 12 millivolts as examples with no limitation intended. In an example, the ventricular sensing thresholds 326 and 334 may be programmable in the range of 0.45 mV to 11.3 mV. The absolute values of the amplitudes of the positive and negative first and second ventricular sensing threshold amplitudes 326 and 334 may be different from each other and may be individually programmable by a user.

In the example shown, sensing circuit 204 determines that the ventricular EGM signal 322 meets a requirement relating to the second ventricular sensing threshold 334 when the ventricular EGM signal 322 crosses the second ventricular sensing threshold 334 prior to the expiration of the Vsense confirmation window 332. In this case, the ventricular sensing electrode vector may be positioned such that a substantially biphasic R-wave 336 is expected to have a predominate negative going peak followed by a positive going peak. Therefore sensing circuit 204 may apply a negative first ventricular sensing threshold 326 followed by a positive second ventricular sensing threshold 334 for sensing R-wave 336. The far field P-wave 324 in the ventricular EGM signal may be monophasic or biphasic and may or may not be smaller in amplitude relative to the R-wave 336. The first ventricular sensing threshold 326 and the requirement relating to the second ventricular sensing threshold requirement 334 are selected to provide discrimination between R-wave 336 and far field P-wave 324 in the ventricular EGM signal 322 based on amplitude and polarity of the R-wave 336 relative to the amplitude and polarity of the far-field P-wave 324 in the ventricular EGM signal 322. In other examples, no crossing of the second ventricular sensing threshold 334 during the Vsense confirmation window 332 may be required for the sensing circuit 204 to determine that the requirement relating to the second ventricular sensing threshold is met for confirming threshold crossing 330 as being an R-wave. In this case, the ventricular sensing electrode vector may be positioned such that a negative-going, monophasic R-wave is expected in the ventricular EGM signal 322.

When the second ventricular sensing threshold requirement is met by the expiration of the Vsense confirmation window 332, sensing circuit 204 may generate the Vsense signal 338. The Vsense signal 338 may be passed to control circuit 206, optionally including a time stamp corresponding to the time of the first ventricular sensing threshold crossing 338 (as shown) or corresponding to the time of the second ventricular sensing threshold crossing 331 when the Vsense is confirmed. In other examples, the Vsense signal 338 may be generated upon expiration of the Vsense confirmation window 332. Control circuit 206 may optionally adjust the time of the ventricular sensed event by the time duration of the Vsense confirmation window 332. For example, the duration of the Vsense confirmation window 332 may be subtracted from the time expired on an escape interval timer included in pace timing circuit 242 (FIG. 3) since a most recent preceding Vsense signal or delivered ventricular pacing pulse for determining a ventricular event interval and a corresponding ventricular rate.

In other examples, sensing circuit 204 may additionally or alternatively determine the absolute value of the maximum peak amplitude 335 of the ventricular EGM signal 322 during the Vsense confirmation window 332. Sensing circuit 204 may determine the absolute value of the maximum peak amplitude 315 of the atrial EGM signal 302 during the Vsense confirmation window 332. Sensing circuit 204 may verify that the ventricular EGM maximum peak amplitude 335 is greater (in absolute value) than atrial EGM maximum peak amplitude 315 to confirm the likelihood of threshold crossing 330 being associated with an R-wave 336 and not a far field P-wave. If threshold crossing 330 is caused by a far field P-wave, the near field P-wave in the atrial EGM signal 302 may be expected to have a greater maximum peak amplitude during the Vsense confirmation window 332 than the maximum peak amplitude of the far field P-wave in ventricular EGM signal 322.

Sensing circuit 204 may apply the first ventricular sensing threshold 330 without applying a second ventricular sensing threshold in some examples. Instead, sensing circuit 204 may verify that an atrial sensing threshold crossing by the atrial EGM signal 302 does not occur during the Vsense confirmation window 332, confirm that an Asense confirmation window is not running at the time of the first ventricular sensing threshold crossing 330, and/or confirm that the absolute maximum peak amplitude 335 of ventricular EGM signal 322 is greater than the absolute maximum peak amplitude 315 of atrial EGM signal 302 sensed during the Vsense confirmation window 332.

As generally described above, comparison of the absolute maximum peak amplitudes 315 and 335 is one example of a comparison that can be made by sensing circuit 204 (or control circuit 206) between atrial EGM signal 302 and ventricular EGM signal 322 for verifying that the first ventricular sensing threshold crossing 330 is likely an R-wave and generating a Vsense signal 338. In other examples, a maximum positive slope, maximum negative slope, minimum peak amplitude, maximum peak amplitude, peak-to-peak amplitude, signal width, number of zero crossings, number of peaks (positive and/or negative), signal area, and/or other feature(s) determined from atrial EGM signal 302 and ventricular EGM signal 322 sensed during the Vsense confirmation window 332 may be determined and compared by sensing circuit 204 and/or control circuit 206 for verifying that R-wave sensing criteria are met.

When R-wave sensing criteria are met during or by the expiration of the Vsense confirmation window 332, sensing circuit 204 may generate the Vsense signal 338 for passing to control circuit 206. Control circuit 206 may adjust the timing of the Vsense signal 338 if the Vsense confirmation window 332 has caused a delay in generating the Vsense signal 338 from the actual time of the first sensing threshold crossing 330 in some examples. In other examples, the Vsense signal 338 may be generated as a pending Vsense signal by sensing circuit 204 upon the first ventricular sensing threshold crossing 330 for use as a Vsense timing marker by control circuit 206. Subsequently, in some examples sensing circuit 204 may generate a Vsense confirmation signal 339 in response to the second ventricular sensing threshold crossing 331. In response to receiving the Vsense confirmation signal 339, control circuit 206 may use the Vsense signal 338 (and/or the Vsense confirmation signal 339) for starting various timing control intervals, e.g., a post-ventricular atrial blanking period, a ventricular refractory period, a ventricular blanking period and a ventricular pacing interval. Control circuit 206 may use the Vsense signal 338 and/or Vsense confirmation signal 339 for determining a ventricular event interval or RRI, e.g., based on a ventricular event interval timer. Control circuit 206 may ignore the Vsense signal 338 if the Vsense confirmation signal 339 is not received at the expiration of the Vsense confirmation window 332. In some examples, control circuit 206 may use the timing of the Vsense confirmation signal 339 for determining an RRI and/or starting one or more timing control intervals. A time stamp of a confirmed Vsense signal 338 and/or the Vsense confirmation signal 339 may be stored in memory 210 for use by control circuit 206 in determining ventricular event intervals (e.g., RRIs) and corresponding ventricular rate.

If the Vsense signal 338 is not confirmed at the expiration of the Vsense confirmation window 332, any currently running timing control intervals may continue running. The Vsense signal 338 may be ignored for the purposes of resetting post-ventricular sense timing control intervals. If an AV pacing interval or a ventricular pacing escape interval expires during the Vsense confirmation window 332, a pending ventricular pacing pulse may be delayed until the expiration of the Vsense confirmation window 332. If the Vsense signal 338 is confirmed, the pending ventricular pacing pulse may be cancelled. If the Vsense signal 338 is not confirmed, the pending ventricular pacing pulse may be delivered at the expiration of the Vsense confirmation window 338. In some cases, a ventricular pacing pulse may be delivered upon expiration of an AV pacing interval during a Vsense confirmation window 332 to promote AV synchrony. If the pending Vsense 338 is false, the ventricular pacing pulse is needed. If the pending Vsense 338 is true, a delivered ventricular pacing pulse may fall during physiological refractory or result in fusion pacing of the ventricle.

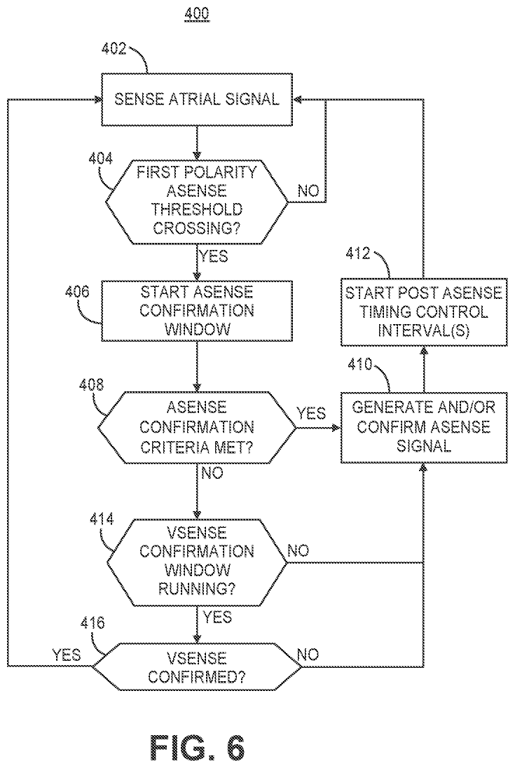

FIG. 6 is a flow chart 400 of a method that can be performed by pacemaker 14 for sensing P-waves according to some examples. The flow chart 400 and other flow charts and diagrams presented herein may be performed cooperatively by sensing circuit 204 and control circuit 206. Sensing circuit 204 may receive various sensing control parameters from control circuit 206 and pass sensed event signals and/or sensed event confirmation signals to control circuit 206. In some examples, sensing circuit 204 may pass sensed EGM signals to control circuit 206 for analysis by control circuit 206 for confirming a received Asense or Vsense signal. It is to be understood, therefore, that aspects of the techniques disclosed herein for sensing cardiac event signals, e.g., P-waves and/or R-waves, may be performed by control circuit 206 configured to receive at least one digitized, non-rectified cardiac electrical signal from sensing circuit 204. As such, the terms “sensing circuitry” and “control circuitry” as used herein may refer to circuitry configured to perform functions attributed to sensing circuit 204 and/or control circuit 206 as described herein. The operations performed for sensing and confirming P-waves and/or R-waves and starting various timing control intervals based on confirmed sensed P-waves and/or R-waves may be performed cooperatively by sensing circuitry and control circuitry of pacemaker 14 that can be functionally represented as sensing circuit 204 and/or control circuit 206 in FIG. 3.

At block 402 of FIG. 6, an atrial signal is sensed by sensing circuit 204 using a selected atrial sensing electrode vector coupled to the sensing circuitry. As described above, the atrial signal may be filtered, amplified and digitized by an atrial sensing channel 203 for passing a non-rectified atrial EGM signal to P-wave detection circuitry, e.g., included in atrial event detector circuit 226 (FIG. 4). At block 404, sensing circuit 204 may determine if the non-rectified atrial EGM signal crosses a first polarity atrial sensing threshold outside of any atrial blanking periods. The first polarity atrial sensing threshold may have a positive or negative amplitude. Sensing circuit 204 continues sensing the atrial signal at block 402 until a crossing of the first polarity atrial sensing threshold is detected at block 404.

At block 406, sensing circuit 204 may start an Asense confirmation window as described above in conjunction with FIG. 5A in response to detecting the first polarity atrial sensing threshold crossing at block 404. At block 408, the sensing circuitry may determine if P-wave sensing criteria are met based on the atrial EGM signal and/or the ventricular EGM signal sensed during the Asense confirmation window.

In some examples, sensing circuit 204 may determine if a requirement relating to a second polarity atrial sensing threshold is met at block 408. The second polarity atrial sensing threshold has the opposite polarity of the first polarity atrial sensing threshold. As described above, the second polarity atrial sensing threshold requirement may be met when the second polarity atrial sensing threshold is crossed by the atrial EGM signal before the expiration of the Asense confirmation window. In other examples, the requirement relating to the second polarity atrial sensing threshold requirement may be met when the atrial EGM signal does not cross the second polarity atrial sensing threshold before the expiration of the Asense confirmation window.

If the second polarity atrial sensing threshold requirement is met, the sensing circuit 204 may generate an Asense signal at block 410. As described above, in other examples, an Asense signal may be generated by the atrial sensing channel 203 in response to the first polarity atrial sensing threshold crossing at block 404. Sensing circuit 204 may then generate an Asense confirmation signal at block 410 when the second polarity atrial sensing threshold requirement is met. In still other examples, control circuit 206 may receive a pending Asense signal from sensing circuit 204 at block 404 when the first polarity atrial sensing threshold is crossed and receive the atrial EGM signal sensed during the Asense confirmation window. Control circuit 206 may determine when a second polarity atrial sensing threshold requirement is met at block 408 and thereby confirm the validity of the received pending Asense signal.

Additionally or alternatively, the sensing circuitry may determine that the P-wave sensing criteria are met at block 408 based on a comparison of one or more features of the atrial EGM signal to an analogous feature of the ventricular EGM signal sensed during the Asense confirmation window. Examples of atrial EGM and ventricular EGM signal features that may be determined and compared are listed above in conjunction with FIG. 5A. In some examples, the maximum absolute amplitude, maximum positive amplitude, minimum negative amplitude, and/or peak-to-peak amplitude of the atrial EGM signal may be determined and compared to the analogous maximum absolute amplitude maximum positive amplitude, minimum negative amplitude, and/or peak-to-peak amplitude of the ventricular EGM signal sensed during the Asense confirmation window. For instance, when the maximum absolute amplitude of the atrial EGM signal sensed during the Asense confirmation window is greater than the absolute amplitude of the ventricular EGM signal sensed during the Asense confirmation window, the sensing circuitry may determine that the P-wave sensing criteria are met at block 408.

When the P-wave sensing criteria are met by the atrial EGM signal sensed during the Asense confirmation window (and in some cases by the ventricular EGM signal sensed during the Asense confirmation window), sensing circuit 204 and/or control circuit 206 may start one or more post-atrial sense timers at block 412 according to one or more timing control intervals. An atrial blanking period, an atrial refractory period, an atrial event interval timer, a post-atrial ventricular blanking period, an atrial pacing escape interval, and/or an AV pacing interval are examples of timing control intervals that may be started by pacemaker control circuitry in response to confirming an Asense signal.

Referring again to block 408, when the second polarity atrial sensing threshold requirement or other P-wave sensing criteria are not met, sensing circuit 204 may determine if a Vsense confirmation window is running at block 414 during any portion of the Asense confirmation window or at the expiration of the Asense confirmation window. If a Vsense confirmation window is not running, the Asense signal may be confirmed at block 410 based on the first polarity atrial sensing threshold crossing. A cross-chamber oversensed far field R-wave may be unlikely if the Vsense confirmation window has not been started prior to the expiration of the Asense confirmation window.

If sensing circuit 204 determines that a Vsense confirmation window is running at block 414, the first polarity atrial sensing threshold crossing may be caused by a far field R-wave in the atrial EGM signal. The Vsense confirmation window may be started in response to a first polarity ventricular sensing threshold crossing by a sensed ventricular EGM signal and may be running during and/or at the expiration of the Asense confirmation window. Sensing circuit 204 may withhold or delay confirming an Asense signal based on the first polarity atrial sensing threshold crossing if a Vsense confirmation window is running during the Asense confirmation window.

In the example shown in FIG. 6, sensing circuit 204 may determine if a Vsense signal is confirmed based on at least the ventricular EGM signal sensed during the Vsense confirmation window (and in some examples in combination with the atrial EGM signal sensed during the Vsense confirmation window) at block 416. If a Vsense signal is not confirmed by sensing circuit 204 (and/or control circuit 206) at block 416, the Asense signal may still be confirmed at block 410 based on the first polarity atrial sensing threshold crossing and a Vsense signal not being confirmed. Post-atrial sense timing control intervals may be started by sensing circuit 204 and/or control circuit 206 at block 412 using an effective starting time of the first polarity Asense threshold crossing or the expiration time of the Asense confirmation window.

When a Vsense signal is confirmed by the sensing circuitry at block 416, e.g., based on at least the ventricular EGM signal sensed during the Vsense confirmation window, the sensing circuitry does not confirm the Asense signal. The process may return to block 402 (“yes” branch of block 416) without starting any post-atrial timing control intervals. In other examples, the sensing circuitry may not wait to determine if a Vsense signal is confirmed at block 416 after determining that a Vsense confirmation window is running during or at the expiration of the Asense confirmation window at block 414. The process of flow chart 400 may return to block 402 without confirming the Asense signal by the sensing circuitry. Sensing circuit 204 may ignore the first polarity atrial sensing threshold crossing for the purposes of starting timing control intervals, e.g., by not confirming the Asense signal due to the running Vsense confirmation window during or at the expiration of the Asense confirmation window.

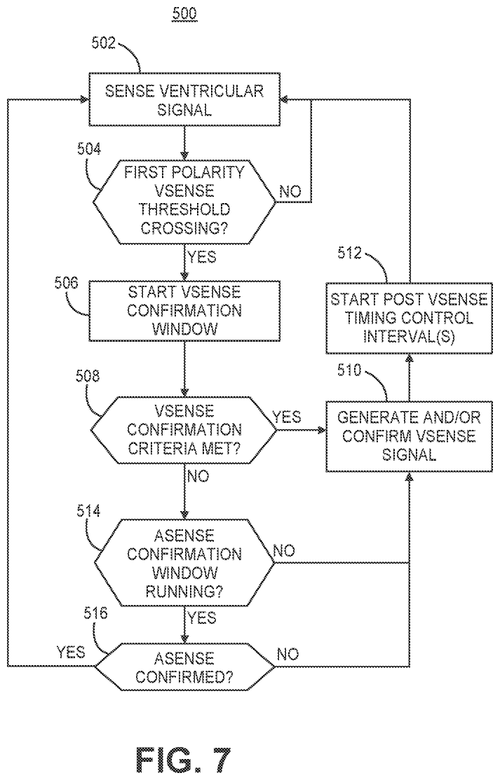

FIG. 7 is a flow chart 500 of a method that can be performed by pacemaker 14 for sensing R-waves according to some examples. The flow chart 500 may be performed cooperatively by sensing circuit 204 and control circuit 206. Sensing circuit 204 may receive various sensing control parameters from control circuit 206 and pass sensed event signals and, in some examples, sensed event confirmation signals to control circuit 206. In some examples aspects of the process of flow chart 500 for confirming a Vsense signal received from sensing circuit 204 may be performed by control circuit 206 configured to receive at least one digitized, non-rectified cardiac electrical signal from sensing circuit 204. As such, functionality of sensing circuit 204 and control circuit 206 may be referred to as the “sensing circuitry” performing the cardiac event sensing techniques disclosed herein.

At block 502 of FIG. 7, a ventricular signal is sensed by sensing circuit 204 using a selected ventricular sensing electrode vector coupled to the sensing circuit 204. As described above, the received ventricular signal may be filtered, amplified and digitized by a ventricular sensing channel 205 for passing a non-rectified ventricular EGM signal to R-wave detection circuitry, e.g., included in ventricular event detector circuit 256 (FIG. 4). At block 504, sensing circuit 204 may determine if the non-rectified ventricular EGM signal crosses a first polarity ventricular sensing threshold outside of any ventricular blanking periods. The first polarity ventricular sensing threshold may be a positive or negative sensing threshold amplitude. Sensing circuit 204 continues sensing the ventricular signal at block 502 until a crossing of the first polarity ventricular sensing threshold is detected at block 504.

At block 506, sensing circuit 204 may start a Vsense confirmation window as described above in conjunction with FIG. 5B in response to detecting the first polarity ventricular sensing threshold crossing. At block 508, sensing circuit 204 may determine if R-wave sensing criteria are met during or by the expiration of the Vsense confirmation window. In some examples, sensing circuit 204 may determine if requirement relating to a second polarity ventricular sensing threshold is met. As described above, the second polarity ventricular sensing threshold requirement may be met when a second polarity ventricular sensing threshold crossing occurs during the Vsense confirmation window. In other examples, the second polarity ventricular sensing threshold requirement may be met when the ventricular EGM signal does not cross the second polarity ventricular sensing threshold during the Vsense confirmation window. The amplitude of the second polarity ventricular sensing threshold has the opposite polarity of the amplitude of the first polarity ventricular sensing threshold. The amplitudes may be the same or different.

Additionally or alternatively, the sensing circuitry may determine that the R-wave sensing criteria are met at block 508 based on a comparison of one or more features of the ventricular EGM signal to an analogous feature of the atrial EGM signal sensed during the Vsense confirmation window. Examples of ventricular EGM and atrial EGM signal features that may be determined and compared are listed above in conjunction with FIG. 5B. In various examples, the maximum absolute amplitude, maximum positive amplitude, minimum negative amplitude and/or peak-to-peak amplitude of the ventricular EGM signal sensed during the Vsense confirmation window may be determined and compared to the analogous maximum absolute amplitude, maximum positive amplitude, minimum negative amplitude, and/or peak-to-peak amplitude of the atrial EGM signal sensed during the Vsense confirmation window. For instance, when the maximum absolute amplitude of the ventricular EGM signal is greater than the absolute amplitude of the atrial EGM signal sensed during the Vsense confirmation window, the sensing circuitry may determine that the R-wave sensing criteria are met at block 508.

Sensing circuit 204 may additionally determine whether a post-atrial ventricular blanking period (PAVBP) is running at the expiration of the Vsense confirmation window. R-wave sensing criteria applied at block 508 may include requiring that a PAVBP is not running at the expiration of the Vsense confirmation window. If a PAVBP is running at the expiration of the Vsense confirmation window, sensing circuit 204 may determine that R-wave sensing criteria are not met at block 508. The first polarity ventricular sensing threshold crossing detected at block 504 may be caused by a far field P-wave present in the ventricular EGM signal. As such, sensing circuit 204 may not confirm a Vsense signal due to the running PAVBP.

If the second polarity ventricular sensing threshold requirement and/or other R-wave sensing criteria is/are met at block 508, the sensing circuit 204 may generate a Vsense signal at block 510. In other examples, a Vsense signal may be generated by the ventricular sensing channel 205 in response to the first polarity ventricular sensing threshold crossing at block 504. Sensing circuit 204 may then generate a Vsense confirmation signal at block 510 when the R-wave sensing criteria are met. In still other examples, control circuit 206 may receive a pending Vsense signal from sensing circuit 204 at block 504 when the first polarity ventricular sensing threshold is crossed and receive the ventricular EGM signal and the atrial EGM signal sensed during the Vsense confirmation window from sensing circuit 204. Control circuit 206 may determine when a second polarity ventricular sensing threshold requirement and/or other R-wave sensing criteria is/are met at block 508 and thereby confirm the pending Vsense signal at block 510.

When the R-wave sensing criteria are met, sensing circuit 204 and/or control circuit 206 may start one or more post-ventricular sense timers at block 512 according to one or more timing control intervals. A ventricular blanking period, a ventricular refractory period, a post-ventricular atrial blanking period, a post-ventricular atrial refractory period, a ventricular event interval timer, and/or a ventricular pacing escape interval may be started in response to a confirmed Vsense signal, as examples.

Referring again to block 508, when a requirement relating to the second polarity ventricular sensing threshold requirement and/or other R-wave sensing criteria is/are not met, the first polarity ventricular sensing threshold crossing may be an oversensed T-wave present in the ventricular EGM signal. In some examples, if the R-wave sensing criteria are not met at block 508, a Vsense event is not confirmed because the first polarity Vsense threshold crossing could be due to a T-wave or a far field P-wave (or other non-cardiac noise artifacts). The process may return to block 502 from block 508 when the sensing circuitry determines that the R-wave sensing criteria are not met.

In the example shown in FIG. 7, however, sensing circuit 204 may determine if an Asense confirmation window is running during any portion of the Vsense confirmation window or at the expiration of the Vsense confirmation window at block 514. If an Asense confirmation window is not running, the Vsense signal may still be confirmed at block 510 based on the first polarity ventricular sensing threshold crossing and no Asense confirmation window running during the Vsense confirmation window. A cross-chamber oversensed far field P-wave may be unlikely if the Asense confirmation window has not been started or not already running during the Vsense confirmation window.

If sensing circuit 204 determines that an Asense confirmation window is running at block 514, e.g., started in response to a first polarity atrial sensing threshold crossing by a sensed atrial EGM signal, sensing circuit 204 may withhold or delay confirmation of a Vsense signal. Confirmation of a Vsense signal based on the first polarity ventricular sensing threshold crossing may be withheld and the process may return to block 502 without starting any post-ventricular sense timing control intervals. In other examples, as shown in FIG. 7, the sensing circuitry may delay confirmation of the Vsense signal at least until the Asense confirmation window is expired or an Asense signal is confirmed. Sensing circuit 204 may determine if an Asense signal is confirmed based on the atrial EGM signal sensed during the Asense confirmation window at block 516, e.g., based on the methods described above in conjunction with FIG. 6. If an Asense signal is not confirmed by sensing circuit 204 (and/or control circuit 206) at block 516, the Vsense signal may be confirmed at block 510 based on the first polarity ventricular sensing threshold crossing and an Asense signal not being confirmed. Post-ventricular sense timing control intervals may be started at block 512, having an effective starting time coinciding with the time of the first polarity ventricular sensing threshold crossing or the expiration of the Vsense confirmation window. When an Asense signal is confirmed by sensing circuit 204 and/or control circuit 206 at block 516, sensing circuit 204 (and/or control circuit 206) does not confirm the Vsense signal. The process may return to block 502 (“yes” branch of block 516) without starting or applying any post-ventricular timing control intervals.

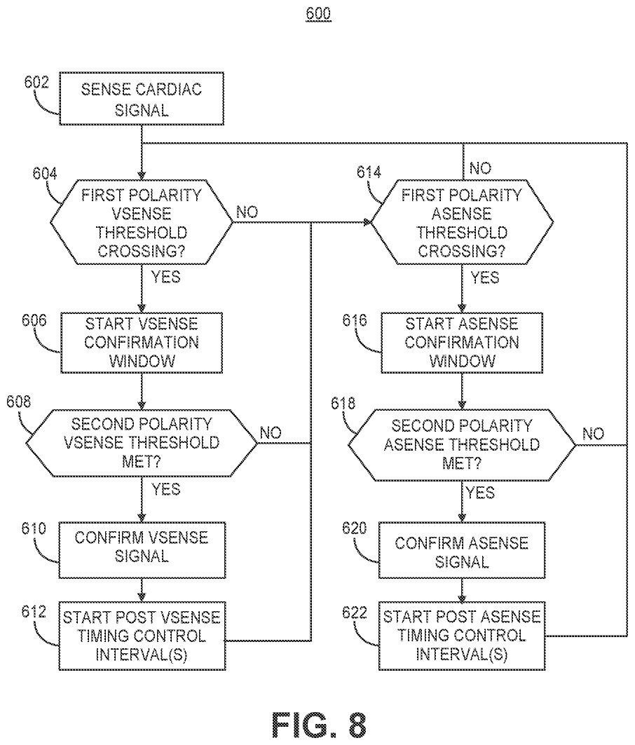

FIG. 8 is a flow chart 600 of a method that may be performed by sensing and control circuitry of pacemaker 14 for sensing cardiac event signals according to another example. It is contemplated that pacemaker sensing circuitry could apply two different polarity atrial sensing thresholds and two different ventricular sensing thresholds to the same EGM signal for separately sensing and discriminating P-waves and R-waves. In this example, at block 602, sensing circuit 204 receives a cardiac electrical signal at block 602 that may be filtered, amplified and digitized without rectification to pass a non-rectified EGM signal to a cardiac event detection circuit of sensing circuit 204 and/or control circuit 206, which may be a single cardiac event detection circuit or processing circuitry configured to apply both atrial and ventricular sensing thresholds or two circuits or processors configured to each apply the atrial or ventricular sensing thresholds.

At block 604, the sensing circuitry may apply a first polarity ventricular sensing threshold to the EGM signal. If the EGM signal crosses the first polarity ventricular sensing threshold outside a ventricular blanking period or post-atrial ventricular blanking period, the sensing circuitry may start a Vsense confirmation window at block 606. The sensing circuitry may determine if a requirement relating to a second polarity ventricular sensing threshold is met by the EGM signal sensed during the Vsense confirmation window at block 608. As described above, the second polarity ventricular sensing threshold is an amplitude having the opposite polarity (positive or negative) of the amplitude (negative or positive) of the first polarity ventricular sensing threshold. In some cases, the requirement relating to the second ventricular sensing threshold requires that the second ventricular sensing threshold is not crossed by the EGM signal during the Vsense confirmation window. In other examples, the requirement relating to the second ventricular sensing threshold requires that the second ventricular sensing threshold is crossed by the EGM signal during the Vsense confirmation window.

In response to the second polarity ventricular sensing threshold requirement being met at block 608, a Vsense signal is confirmed at block 610. Control circuitry of the pacemaker 14 may start one or more post-ventricular sense timing control intervals, e.g., by starting one or more timers according to a ventricular blanking period, post-ventricular atrial blanking period, post-ventricular atrial refractory period, ventricular refractory period, ventricular event interval timer or ventricular pacing interval. A scheduled ventricular pacing pulse may be cancelled by restarting a ventricular pacing interval. The control circuitry may determine the time expired on a ventricular pacing escape interval timer upon confirming the Vsense signal for determining a ventricular sensed event interval or corresponding ventricular rate.

The sensing circuitry applies a first polarity atrial sensing threshold to the sensed EGM signal at block 614. While the flow chart 600 depicts application of the first polarity ventricular sensing threshold first (at block 604) followed by applying the first polarity atrial sensing threshold (at block 614) it is to be understood that, outside of applicable blanking periods, the sensing circuitry may be applying the first polarity ventricular sensing threshold and the first polarity atrial sensing threshold to the EGM signal simultaneously.

In some cases, the first polarity ventricular sensing threshold may have a polarity that is opposite the first polarity atrial sensing threshold such that the two different sensing thresholds cannot be crossed simultaneously or near simultaneously by the EGM signal. In other cases, the polarities may be the same but the amplitudes of the first polarity ventricular sensing threshold and the first polarity atrial sensing threshold may be different such that the EGM signal may cross a lower one of the first polarity ventricular sensing threshold or the first polarity atrial sensing threshold but not both. In some instances, however, when the polarities are the same but the amplitudes are the same or different, a cardiac event signal (P-wave or R-wave) may cross both the first polarity ventricular sensing threshold and the first polarity atrial sensing threshold at or near the same time. As such, the Vsense confirmation window and the Asense confirmation window may both be started such that either a Vsense or an Asense signal may be confirmed based on the requirements relating to one of the second polarity ventricular sensing threshold or the second polarity atrial sensing threshold being met (but not both).

While block 614 is shown successively following block 612 in FIG. 8, therefore, it is to be understood that application of the first ventricular sensing threshold and first atrial sensing threshold may be occurring simultaneously outside of respective blanking periods. The second polarity ventricular sensing threshold and the second polarity atrial sensing threshold may be applied simultaneously when the Vsense confirmation window and the Asense confirmation window overlap. However, the combination of the first polarity ventricular sensing threshold and the requirement relating to the second polarity ventricular sensing threshold is defined distinctly from the combination of the first polarity atrial sensing threshold and the requirement relating to the second polarity atrial sensing threshold so that only one combination can become satisfied at a time.

When the first polarity atrial sensing threshold is crossed by the EGM signal outside any applicable atrial blanking periods, the sensing circuitry may start an Asense confirmation window at block 616. The sensing circuitry determines if a requirement relating to the second polarity atrial sensing threshold is met by the EGM signal sensed during the Asense confirmation window at block 618. If not, the sensing circuitry may return to block 604 to wait for a first polarity ventricular sensing threshold crossing and/or a first polarity atrial sensing threshold crossing.

When the requirement relating to the second polarity atrial sensing threshold is met at block 618, the sensing circuitry may confirm the Asense signal at block 620. As described above, the second polarity atrial sensing threshold requirement may be met when the EGM signal crosses the second polarity atrial sensing threshold during the Asense confirmation window. In other examples, the second polarity atrial sensing threshold requirement may be met when the EGM signal does not cross the second polarity atrial sensing threshold during the Asense confirmation window. In response to the Asense signal being confirmed at block 620, pacemaker control circuitry may start one or more post-atrial sense timing control intervals at block 622, e.g., an atrial blanking period, atrial refractory period, post-atrial ventricular blanking period, atrial event interval timer, atrial pacing interval and/or AV pacing interval. A scheduled atrial pacing pulse may be cancelled by restarting the atrial pacing interval. The process may then return to block 604 to wait for a subsequent crossing of the first polarity ventricular sensing threshold or the first polarity atrial sensing threshold by the EGM signal.

In this way, two different polarity ventricular sensing thresholds and two different polarity atrial sensing thresholds may be defined with a Vsense confirmation requirement relating to the second polarity ventricular sensing threshold and an Asense confirmation requirement relating to second polarity atrial sensing threshold that exclusively define R-wave sensing criteria and P-wave sensing criteria. Both criteria are highly unlikely to be satisfied by the same cardiac event signal. For instance, even if both of the first polarity ventricular sensing threshold and the first polarity atrial sensing threshold could be crossed by an initially positive or negative going waveform of the EGM signal, the requirements defined relating to the second polarity sensing thresholds may be mutually exclusive. The mutually exclusive dual polarity sensing threshold criteria enables the sensing circuitry to distinguish between P-waves and R-waves (and T-waves) in an EGM signal.

In an illustrative scenario, the first polarity ventricular sensing threshold amplitude may be the same polarity but greater in amplitude than the first polarity atrial sensing threshold. In some instances, the EGM signal may cross the first polarity atrial sensing threshold but not the first polarity ventricular sensing threshold. If the requirement relating to the second polarity atrial sensing threshold is met, the Asense signal is confirmed. However, if the first polarity ventricular sensing threshold is also crossed by the same signal as the first polarity atrial sensing threshold, the requirements relating to the second polarity ventricular sensing threshold and the second polarity atrial sensing threshold may be exclusive, even if the second polarities are the same (e.g., both positive amplitudes or both negative amplitudes). For instance, the requirement relating to the second polarity ventricular sensing threshold may require that the EGM signal cross the second polarity ventricular sensing threshold and the requirement relating to the second polarity atrial sensing threshold may require that the EGM signal does not cross the second polarity atrial sensing threshold. The combination of the first and second polarity ventricular sensing thresholds and the first and second polarity atrial sensing thresholds can be mutually exclusive in this way to enable reliable sensing and discrimination of P-waves and R-waves from a single or two different EGM signals while reliably avoiding T-wave oversensing.

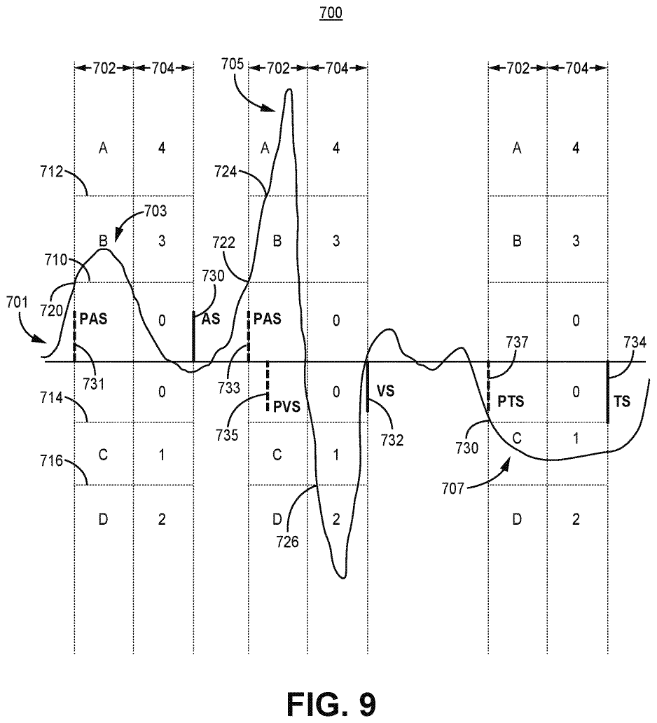

FIG. 9 is a diagram 700 of example thresholds and time windows that may be applied to a sensed cardiac electrical signal for sensing and discriminating P-waves, R-waves and, if desired, T-waves, according to some examples. In this example, cardiac electrical signal 701 is sensed be a sensing electrode vector selected for use in sensing both P-waves, R-waves and, if desired, T-waves. In this example, a first positive threshold amplitude 710, a second positive threshold amplitude 712, a first negative threshold amplitude 714 and/or a second negative threshold amplitude 716 may be applied to the incoming cardiac electrical signal 701 by a cardiac event detector circuit of sensing circuitry of pacemaker 14.

When the first positive threshold 710 or the first negative threshold 714 is crossed by the cardiac electrical signal 701 outside any applied blanking periods, a first confirmation window 702 is started by the sensing circuitry of pacemaker 14. The sensing circuitry may determine if cardiac electrical signal 701 crosses the second positive threshold 712 or the second negative threshold 716 within the first confirmation window 702 after a respective first positive threshold crossing 720 or a first negative threshold crossing 730.

After crossing the first positive sensing threshold amplitude 710, the cardiac electrical signal 701 may cross the second positive sensing threshold amplitude 712 into an amplitude zone A. This occurs in this example when R-wave 705 crosses the first positive threshold amplitude 710 at threshold crossing 722 and crosses the second positive threshold amplitude 712 at crossing 724 within the first confirmation window 702.

In other instances, after crossing the first positive sensing threshold amplitude 710, the cardiac electrical signal 701 may reach a maximum peak that is in the amplitude zone B between the two positive sensing threshold amplitudes 710 and 712. This occurs when P-wave 703 crosses the first sensing threshold amplitude 710 at threshold crossing 720 but does not cross the second threshold amplitude 712 during the first confirmation window 702.

After crossing the first negative sensing threshold amplitude 714, the cardiac electrical signal 701 may or may not cross the second negative sensing threshold amplitude 716 within the first confirmation window 702. The minimum negative peak may fall in amplitude zone C between the first and second negative threshold amplitudes 714 and 716, as shown by T-wave 707, or may fall in amplitude zone D, amplitudes less than the second negative threshold amplitude 716.

In some examples, the sensing circuitry applies the positive sensing threshold amplitudes 710 and 712 to detect positive-going crossings, e.g., threshold crossing 720 or threshold crossing 724. Negative-going crossings of the positive sensing threshold amplitudes 710 and 712 by non-rectified cardiac electrical signal 701 may be ignored. Similarly, the sensing circuitry 204 applies the negative sensing threshold amplitudes 714 and 716 to detect negative-going crossings, e.g., threshold crossing 730. Positive-going crossings of the negative sensing threshold amplitudes 714 and 716 by cardiac electrical signal 701 may be ignored. However, R-wave sensing criteria, P-wave sensing criteria, and/or T-wave sensing criteria could conceivably include requirements relating to negative-going crossings of a positive sensing threshold and/or positive-going crossings of a negative sensing threshold within a given confirmation time window in other examples.

The sensing circuitry of pacemaker 14 (or other medical device) may identify an amplitude zone, shown labeled as A, B, C or D in this example, that the amplitude of the sensed cardiac electrical signal 701 reaches during the first confirmation window 702. The amplitude zone may be determined based on whether or not the second positive sensing threshold amplitude 712 is crossed or the second negative sensing threshold amplitude 716 is crossed within the first confirmation window 702. In other examples, a maximum peak amplitude and/or minimum peak amplitude may be determined from the signal 701 sensed during the first confirmation window 702. The maximum and/or minimum peak amplitude may be compared to the sensing threshold amplitudes 710, 712, 714 and 716 for determining an amplitude zone A, B, C or D for the first confirmation window 702.

Upon expiration of the first confirmation window 702, a second confirmation window 704 is started. The sensing circuitry may determine the amplitude zone, which may be 0, 1, 2, 3 or 4 in this example, that the sensed cardiac electrical signal 701 reaches during the second confirmation window 704. The amplitude zone between the first positive sensing threshold amplitude 710 and the first negative sensing threshold amplitude 714 may be labeled “0” in the second confirmation window 704. When the amplitude of the cardiac electrical signal 702 falls below the first positive sensing threshold amplitude 710 before the second confirmation window 704 and no threshold crossings are detected during the second confirmation window 704, the sensing circuitry may determine the amplitude zone to be 0 for the second confirmation window 704. If the cardiac electrical signal 702 crosses the first negative sensing threshold amplitude 714 and does not cross the second negative sensing threshold amplitude 716, the sensing circuitry may determine the amplitude zone labeled “1” for the second confirmation window. If the second negative sensing threshold amplitude 716 is crossed during the second confirmation window 704, the sensing circuitry may determine amplitude zone “2” for the second confirmation window 704.

If the cardiac electrical signal 702 crosses the first positive sensing threshold amplitude 710 and does not cross the second positive sensing threshold amplitude 712 during the second confirmation window 704, the sensing circuitry may determine the amplitude zone labeled “3” for the second confirmation window. If the second positive sensing threshold amplitude 712 is crossed, the sensing circuitry may determine amplitude zone “4” for the second confirmation window 704. In various examples, the amplitude zone determined for a confirmation window 702 or 704 may be determined based on the greatest absolute value of the cardiac electrical signal amplitude during the respective confirmation window. A sensing threshold amplitude may be crossed prior to or during the respective confirmation window such that the highest positive or lowest negative amplitude falls within a given amplitude zone during the confirmation window. For example, the cardiac electrical signal 702 may cross the second sensing threshold amplitude 712 during the first confirmation window and may remain greater than the second sensing threshold amplitude 712 during at least a portion of the second confirmation window 704 resulting in an amplitude zone of A for the first and second confirmation windows.

When the cardiac electrical signal 701 reaches amplitude zone A or B in the first confirmation window 702, the sensing circuitry may apply the first and second negative sensing threshold amplitudes 714 and 716 during the second confirmation window 704. If the first negative sensing threshold amplitude 714 causes the first confirmation window 702 to be started, the sensing circuitry may apply the first and second positive sensing threshold amplitudes 710 and 712 during the second sensing threshold window 704. However, depending on the cardiac event signal(s) being sensed from cardiac electrical signal 701, both of the second positive sensing threshold amplitude 712 and the second negative sensing threshold amplitude 716 may be applied during the second confirmation window 704 for detecting a relatively wide signal that may cross either the first positive or first negative sensing threshold amplitude 710 or 714 to start the first confirmation window 702 but is still increasing in absolute value when the second confirmation window 704 is started.

The first and second confirmation windows 702 and 704 may each be 10 ms to 50 ms or 10 to 25 ms in duration as examples. The first confirmation window 702 and the second confirmation window 704 may each have a programmable duration and may have the same or different durations. The total duration of the first and second confirmation windows 702 and 704 combined may be limited to a maximum duration, e.g., up to 20 ms, up to 30 ms, up to 40 ms, up to 50 ms, up to 60 ms, up to 70 ms, up to 80 ms or up to 100 ms as examples.

The first positive sensing threshold amplitude 710 and the first negative sensing threshold amplitude 714 may have the same or different absolute values. The second positive sensing threshold amplitude 712 and the second negative sensing threshold amplitude 716 may have the same or different absolute values. Each of the sensing threshold amplitudes 710, 712, 714 and 716 may be user programmable or established (and adjusted as needed) by the sensing circuitry based on an analysis of the sensed cardiac electrical signal 701, e.g., based on the absolute maximum peak amplitude of a sensed waveform.

In some examples, the sensing circuitry starts a first confirmation window 702 only in response to a crossing of the first positive sensing threshold amplitude 710. In other examples, the sensing circuitry may start first confirmation windows 702 only in response to a crossing of the first negative sensing threshold amplitude 714. In still other examples, a first confirmation window 702 may be started in response to a crossing of either a positive sensing threshold amplitude 710 or a negative sensing threshold amplitude 714. The polarity and amplitude of the sensing threshold applied to the sensed cardiac electrical signal for starting the first confirmation window 702 can depend on the polarity of each of the cardiac event signals that are being sensed.

For instance, in the example shown, if only P-wave and R-wave sensing is desired and T-wave sensing is to be avoided, only the first positive sensing threshold amplitude 710 may be applied to the cardiac electrical signal 701 outside of any applicable blanking periods to enable sensing of P-wave 703 and R-wave 705 because both waveforms are initially positive in polarity. The T-wave 707 is initially negative in polarity. By not applying a first negative sensing threshold amplitude 714 outside of any blanking periods, T-wave oversensing is avoided. Once the first positive sensing threshold amplitude 710 is crossed, the second positive sensing threshold amplitude 712 can be applied during the first confirmation window 702. At the expiration of the first confirmation window 702, the sensing circuitry may determine the first window amplitude zone as being A or B, based on whether the second positive sensing threshold amplitude 712 is crossed (zone A) or is not crossed (zone B) during the first confirmation window 702.

The first negative sensing threshold amplitude 714 and/or the second negative sensing threshold amplitude 716 may be applied to the sensed cardiac electrical signal 701 during the second confirmation window 704. The first positive sensing threshold crossing 720 can be a confirmed Asense when a crossing of the first negative sensing threshold 714 does not occur during the second confirmation window 704. In this case, the sensing circuitry may determine a combination of B0 for the first window amplitude zone (B) and the second window amplitude zone (0) as meeting P-wave sensing criteria. An Asense signal 730 may be generated by the sensing circuitry at the expiration of the second confirmation window 704 when the B0 combination is determined.

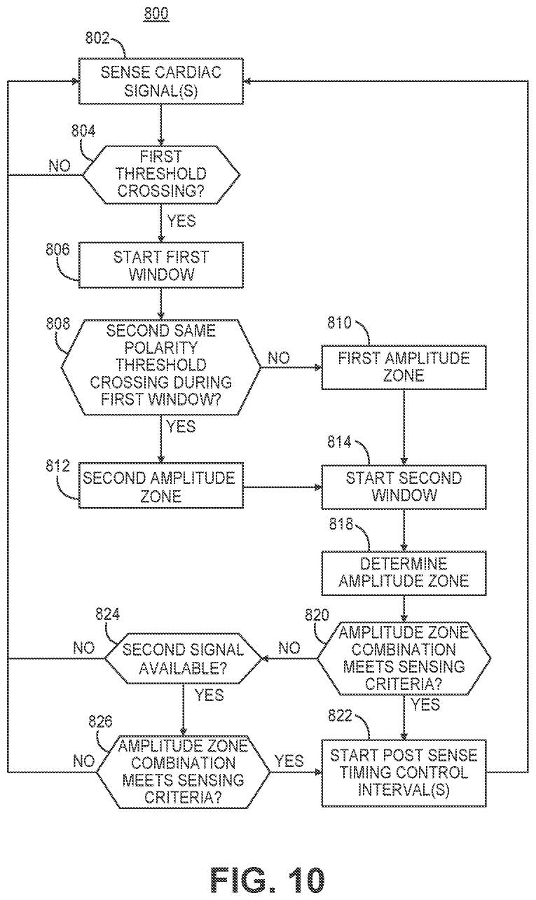

If the first negative sensing threshold 714 is crossed during the second confirmation window, resulting in a combination of B1 or B2, the Asense signal 730 may be withheld due to P-wave sensing criteria not being met. The sensing circuitry may determine if the B1 or B2 combination meets cardiac event sensing criteria to determine if an R-wave or T-wave could be sensed. In the example shown and in Table I below, the B1 and B2 combinations do not correspond to the amplitude and polarity of a P-wave, R-wave or T-wave. The B1 or B2 combination can be determined as an indeterminate signal that could be an unknown cardiac signal, non-cardiac noise or another unknown signal. As described below in conjunction with FIG. 10, in some examples, a second sensed cardiac electrical signal may be analyzed when cardiac event sensing criteria are not met by the first sensed cardiac electrical signal resulting in an indeterminate signal detection based on the first and second confirmation window amplitude zones.

Continuing with the example of FIG. 9, when the second positive threshold amplitude 712 is crossed during the first confirmation window 702 (crossing 724), the sensing circuitry may apply at least the second negative sensing threshold amplitude 716 during the second confirmation window 704. In some examples, both of the first and second negative sensing threshold amplitudes 714 and 716 may be applied. The amplitude zone combination for the first and second confirmation windows 702 and 704 may be A0, A1, or A2 depending on whether a negative sensing threshold amplitude 714 or 716 is crossed. When the sensing circuitry determines an A2 amplitude zone combination, the leading positive sensing threshold crossing 722 may be confirmed to be a true R-wave. The sensing circuitry may generate a Vsense signal 732 at the expiration of the second confirmation window 704.

In other examples, a pending Asense signal (PAS) 731 (or 733) may be generated by the sensing circuitry in response to a first positive sensing threshold crossing 720 (or 722). When P-wave sensing criteria are met at the expiration of the second confirmation window, e.g., in response to a B0 amplitude zone combination, the pending Asense signal 731 may be confirmed. An Asense signal 730 may be generated. Various timing control parameters may be started having an effective starting time at the time of the pending Asense signal 731 or upon expiration of the second confirmation window 704, at the time of the Asense signal 730.

When a pending Asense signal 733 has been generated and the second positive sensing threshold amplitude 712 is crossed during the first confirmation window 702, the pending Asense signal 733 may be canceled. A pending Vsense signal (PVS) 735 may be generated by the sensing circuitry. When the R-wave sensing criteria are met at the expiration of the second confirmation window 704, e.g., when an A2 amplitude zone combination is determined, a Vsense signal 732 may be generated to confirm the pending Vsense signal 735. Various timing control intervals may be started having an effective starting time corresponding to the first sensing threshold crossing 722, the time of the pending Vsense signal 735 or the expiration of the second confirmation window 704.

If an atrial pacing escape interval expires during the first or second confirmation window 702 or 704, and a pending Asense signal 733 has been generated, a pending atrial pacing pulse may be withheld until the expiration of the second confirmation window 704. The pending atrial pacing pulse may be canceled at the expiration of the second confirmation window 704 in response to an Asense signal 733. If no Asense signal is generated, the atrial pacing pulse may be delivered by the pulse generator 202. If an AV pacing interval or a ventricular pacing escape interval expires during the first or second confirmation window 702 or 704, and a pending Asense signal 733 or a pending Vsense signal 735 has been generated, a pending ventricular pacing pulse may be withheld until the expiration of the second confirmation window 704. The pending ventricular pacing pulse may be canceled at the expiration of the second confirmation window 704 in response to a Vsense signal 732. The pending ventricular pacing pulse may be delivered at the expiration of the second confirmation window 704 if the pending Vsense signal 735 is cancelled and no Vsense signal 732 is generated.

In other examples, if an AV pacing interval expires during a confirmation window 702 or 704, a ventricular pacing pulse may be delivered in response to the expiration of the AV pacing interval during a confirmation window to promote AV synchrony. If the pending Vsense signal 735 is true, the delivered atrial synchronous ventricular pacing pulse may result in fusion or be delivered during the ventricular physiological refractory period. If the pending Vsense signal 735 is not true, the delivered ventricular pacing pulse at the AV pacing interval is appropriate for promoting AV synchrony. In still other examples, if an AV pacing interval expires during the first confirmation window 702, the atrial synchronous ventricular pacing pulse may be delivered. However, if the AV pacing interval expires during the second confirmation window 704, the ventricular pacing pulse may be delayed until the expiration of the second confirmation window 704 to avoid pacing into a T-wave if the pending Vsense signal 735 is true. If the Vsense signal 732 is generated, the delayed atrial synchronous ventricular pacing pulse may be cancelled. If the Vsense signal 732 is not generated, the delayed atrial synchronous ventricular pacing pulse can be delivered by the pulse generator 202 at the expiration of the second confirmation window 704.

When the sensing circuitry is configured to sense T-waves, a pending Tsense signal 737 may optionally be generated in response to a first negative sensing threshold crossing 730. A Tsense signal 734 may be generated at the expiration of the second confirmation window 704 when the combination of amplitude zones of the first and second confirmation windows 702 and 704 meet T-wave sensing criteria, e.g., a C1 or C0 amplitude zone combination in this illustrative example. The T-wave morphology may be variable in a given patient and may be a wide negative waveform, wide positive waveform and may be bipolar in some instances. Accordingly, more than one combination of amplitude zones may meet T-wave sensing criteria in some examples.

Table I below is an example list of possible combinations of the first confirmation window amplitude zone and the second confirmation amplitude zone that may be identified by the sensing circuitry once a first confirmation window 702 is started. In this example, the first confirmation window 702 may be started in response to either the first positive or the first negative sensing threshold amplitude. The sensing circuitry may classify a sensed signal according to the amplitude zone combination. Amplitude zone combinations corresponding to P-wave, R-wave and/or T-wave sensing criteria may be programmable by a user. In other examples, amplitude zone combinations may be stored in memory 210 according to fixed sense classifications but the first and second positive sensing threshold amplitudes 710 and 712 and the first and second negative sensing threshold amplitudes 714 and 716 may be user programmable. Additionally or alternatively, the time length (duration) of each of the first confirmation 702 window and the second confirmation window 704 may be user programmable. As seen in Table I, some amplitude zone combinations may be indeterminate. A waveform that crosses a sensing threshold amplitude may be noise or another non-cardiac signal or an indeterminate cardiac signal, such as an ectopic or other aberrantly conducted depolarization.

TABLE I
Example amplitude zone combinations that may be defined
for classifying a cardiac electrical signal waveform.

| Confirmation | Confirmation | |
|---|---|---|
| Window 1 | Window 2 | Sense |
| Amplitude Zone | Amplitude Zone | Classification |
| A | 0 | Indeterminate |
| A | 1 | Indeterminate |
| A | 2 | Vsense |
| A | 3 | Indeterminate |
| A | 4 | T-wave |
| B | 0 | Asense |
| B | 1 | Indeterminate |
| B | 2 | Indeterminate |
| B | 3 | T-wave |
| C | 4 | Indeterminate |
| C | 0 | Indeterminate |
| C | 1 | T-wave |
| C | 2 | Indeterminate |
| C | 3 | Indeterminate |
| C | 4 | Indeterminate |
| D | 0 | Indeterminate |
| D | 1 | Indeterminate |
| D | 2 | T-wave |
| D | 3 | Indeterminate |
| D | 4 | Vsense |

Upon expiration of the second confirmation window 704, the sensing circuitry may determine the first amplitude zone A, B, C or D for the first confirmation window 702 and the second amplitude zone 0, 1, 2, 3 or 4 for the second confirmation window 704. Based on the combination of the first amplitude zone and the second amplitude zone, the sensing circuitry may classify the signal waveform that crossed a first sensing threshold amplitude 710 or 714 as a P-wave, R-wave, T-wave, or an indeterminate signal. It is to be understood, however, that in some examples, not all of the first and second positive and negative threshold amplitudes may be applied in a given first or second confirmation window. The thresholds may be selectively applied according to each amplitude zone combination that positively identifies a cardiac event signal that is to be sensed, e.g., a P-wave, R-wave and/or T-wave.

Furthermore, it is to be understood that the morphology and polarity of each of the P-wave 703, R-wave 705 and T-wave 707 shown in FIG. 9 is one example. The polarity and morphology of each cardiac event signal may vary from patient to patient and in some instances within a given patient due to anatomical variations, relative alignment of the sensing electrode vector to the cardiac axis and direction of propagating signals, inter-electrode spacing of the sensing electrode vector, patient posture, patient physical activity, effects of drugs, and other factors. As such, the number of positive and negative sensing thresholds, their amplitudes, and the corresponding number and possible combinations of amplitude zones for the first and second confirmation windows 702 and 704 may vary between medical device systems and patients and may be programmably tailored to an individual patient. In some cases, multiple combinations of amplitude zones may be defined as meeting sensing criteria for a particular cardiac event signal, e.g., a P-wave, R-wave or a T-wave. The example sense classifications listed in Table I for the possible combinations of confirmation window amplitude zones is therefore illustrative in nature and not intended to be limiting.

Additionally, while only P-waves, R-waves, T-waves or indeterminate signals are listed in Table I and described in conjunction with the accompanying drawings, it is contemplated that one or more combinations of amplitude zones may be defined for sensing non-sinus or aberrantly conducted beats such premature atrial contractions (PACs) and/or premature ventricular contractions (PVCs). A given patient may experience reoccurring PACs and/or PVCs having a common morphology. An amplitude zone combination may be defined to sense a PAC or a PVC that is recurrent in a given patient based on the amplitude zones determined for the first confirmation window and the second confirmation window.

FIG. 10 is a flow chart 800 of a method for sensing cardiac event signals according to some examples. At block 802, the sensing circuitry may receive one or more cardiac electrical signals. In some examples, a single cardiac electrical signal is received for sensing P-waves and/or R-waves (and optionally T-waves) from one signal. In other examples, a second cardiac electrical signal may be sensed for providing a second analysis for sensing and confirming a cardiac event signal when the classification of a sensed waveform is indeterminate based on the analysis of the first cardiac electrical signal.

At block 804, the sensing circuitry waits for a first threshold crossing by the sensed cardiac electrical signal. In some examples, one positive or one negative first sensing threshold amplitude is applied by the sensing circuitry to the cardiac electrical signal outside of any applicable blanking periods (and confirmation windows). In other examples, both a positive and a negative first sensing threshold amplitude may be applied to the cardiac electrical signal outside of any applicable blanking periods.

When a first threshold crossing is detected by the sensing circuitry at block 804, a first confirmation window is started at block 806, e.g., as generally shown by first confirmation window 702 in FIG. 9. During the first confirmation window, the sensing circuitry may apply a second sensing threshold having the same polarity but a greater absolute amplitude as the first sensing threshold that was crossed by the cardiac electrical signal at block 804. When the second, same polarity sensing threshold having a greater absolute amplitude is crossed during the first confirmation window as determined at block 808, the sensing circuitry may determine the corresponding amplitude zone at block 812. For the sake of illustration, if the first threshold crossing is a positive polarity and the second threshold crossing is a higher, positive polarity amplitude crossing, the amplitude zone determined at block 812 is zone A according to the example of FIG. 9, corresponding to relatively large positive amplitude. If a second sensing threshold crossing is not detected during the first confirmation window, a first amplitude zone, e.g., corresponding to relatively small positive amplitude as shown by zone B in the example of FIG. 9, is determined by the sensing circuitry at block 810.

In some examples, one or more sensing amplitude thresholds may be applied during the first confirmation window, each having the same polarity but greater absolute amplitude than the first sensing threshold amplitude that was crossed at block 804. It is contemplated that in some cases a sensing threshold applied during the first confirmation window could be of opposite polarity than the first sensing threshold that was crossed at block 804. However, because the first confirmation window is of relatively short duration, e.g., 10 to 15 ms in some examples, the cardiac electrical signal may be unlikely to reverse polarity and cross an opposite polarity threshold within such a short time interval. The first confirmation window time duration and the sensing threshold(s) applied during the first confirmation window may be selected (e.g., programmed) to enable detection of a depolarization signal is a very narrow biphasic signal, e.g., due to a short inter-electrode distance, relative alignment to the direction of a propagating signal, and/or other factors.

Upon expiration of the first confirmation window, the second confirmation window is started at block 814. During the second confirmation window, the sensing circuitry may apply at least one sensing threshold having a polarity opposite to the first sensing threshold. In some examples, two or more sensing thresholds having different amplitudes and the same and/or opposite polarity as the first sensing threshold crossing detected at block 804 may be applied during the second confirmation window.

Upon expiration of the second confirmation window, the sensing circuitry determines the amplitude zone for the second confirmation window at block 818 based on the applied sensing threshold(s). As shown in the example of FIG. 9, the amplitude zone determined at block 818 may be 0, 1, 2, 3, or 4. In other examples, the amplitude zone may be selected from fewer or more amplitude zones than the five possible zones shown in FIG. 9 depending on how many sensing thresholds are applied at block 818. For example, if a single, negative sensing threshold amplitude is applied during the second confirmation window, the amplitude zone may be either a first or a second amplitude zone depending on whether the single, negative sensing threshold was crossed. The amplitude zone may be determined based on the highest absolute value threshold crossing and its corresponding polarity.

At block 820, the sensing circuitry may determine if cardiac event sensing criteria are met by the combination of amplitude zones determined for the first and second confirmation windows. In some cases, a look up table of amplitude zone combinations, e.g., Table I above, may be stored in memory 210 to enable a sensing classification to be determined from the combination of amplitude zones listed in the look-up table. When sensing criteria are met for a cardiac event signal, e.g., for a P-wave, R-wave or T-wave, the sensing circuitry and/or control circuitry may start any applicable timing control intervals at block 822. The process may return to block 802 to continue sensing the cardiac electrical signal(s) and waiting for the next sensing threshold crossing at block 804.

In some examples, when a second cardiac electrical signal is being sensed by sensing circuit 204, the second cardiac electrical signal may be analyzed in an analogous manner as the first cardiac electrical signal. That is, a first sensing threshold may be applied to the second cardiac electrical signal (block 804). When the first sensing threshold is crossed (block 804), a first confirmation window is started (block 806), and at least one second sensing threshold amplitude, which may be the same polarity as the first sensing threshold, is applied during the first confirmation window. At the expiration of the first confirmation window, a second confirmation window is started (block 814) and one or more sensing thresholds may be applied (block 818), which may include at least one sensing threshold having the opposite polarity as the first sensing threshold that was applied at block 804. At the expiration of the second confirmation window, the amplitude zones determined for the first confirmation window and for the second confirmation window can be determined by the sensing circuitry at block 826.

Referring again to block 820, when cardiac event sensing criteria are not met by the analysis of the first cardiac electrical signal (“no” branch of block 820), e.g., when the classification of the combination of amplitude zones is “indeterminate,” and a second cardiac electrical signal is available (“yes” branch of block 824), a combination of amplitude zones determined from the second cardiac electrical signal may be analyzed at block 826. If cardiac event sensing criteria are met by the amplitude zone combination determined from the second cardiac electrical signal (“yes” branch of block 826), a sensed event signal may be generated and any applicable timing control intervals may be started at block 822. It is to be understood that the number of sensing thresholds and their respective amplitudes and polarities and the number and duration of confirmation windows applied to the second cardiac signal for determining an amplitude zone combination may be different than the number of sensing thresholds and/or their respective amplitudes and polarities and/or the number and/or duration of confirmation windows that are applied to the first cardiac signal. The amplitude zone combinations that meet cardiac event sensing criteria and result in a confirmed sensed cardiac event and/or the amplitude zone combinations that result in an indeterminate signal may be different for the second cardiac signal than the amplitude zone combinations that result in a confirmed sensed cardiac event or indeterminate signal for the first cardiac signal. For example, a different look up table of values may be stored in memory 210 analogous to Table I above for each of the first cardiac signal and the second cardiac signal.

If a second cardiac electrical signal is not available (“no” branch of block 824) or the combination of amplitude zones determined from the second cardiac electrical signal does not meet cardiac event sensing criteria resulting in an indeterminate classification of the sensed waveform (“no” branch of block 826), no sensed cardiac event signal is generated. Timing control intervals that are already running may continue running without interruption and without being reset. The sensing circuitry may return to block 802 to continue sensing the cardiac electrical signal(s) and waiting for the next first sensing threshold crossing at block 804. Any pending cardiac pacing pulse scheduled at a pacing interval that expired during the first or second confirmation window may be delivered at the expiration of the second confirmation window by pulse generator 202.

When a second cardiac electrical signal is available, it may be sensed from a sensing electrode vector having a different inter-electrode spacing and/or different axis relative to the first cardiac electrical signal sensing electrode vector. A combination of amplitude zones and/or any other feature(s) detected from the second cardiac electrical signal may be used for discriminating between P-waves, R-waves, T-waves and/or non-cardiac signals when the analysis of the first cardiac electrical signal is indeterminate.

FIG. 11 is a diagram 900 of confirmation windows and sensing thresholds that may be applied to a cardiac electrical signal for sensing cardiac event signals according to some examples. Cardiac electrical signal 901 includes a P-wave 903, R-wave 905 and T-wave 907. The sensing circuitry may be programmably configured to apply a first positive polarity sensing threshold amplitude 910 outside of any applicable blanking periods. In response to a crossing of the first positive sensing threshold amplitude 910, the sensing circuitry may start a first confirmation window 902 during which the second positive sensing threshold amplitude 912 is applied. At the expiration of the first confirmation window, the sensing circuitry may determine an amplitude zone of either A or B, supporting a pending Vsense or a pending Asense, respectively, based on whether or not the second positive sensing threshold amplitude 912 is crossed.

At the expiration of the first confirmation window 902, the second confirmation window 904 is started. In response to an amplitude zone B determined from the first confirmation window, indicative of a possible P-wave, the sensing circuitry may be programmably configured to apply at least one negative sensing threshold during the second confirmation window 904, e.g., the first negative sensing threshold amplitude 914. The possible amplitude zones that may be determined for the second confirmation window may be 0 or 1, either greater than or less than the first negative sensing threshold amplitude 914 respectively.

In some examples, if the first negative sensing threshold amplitude 914 is not crossed, resulting in a combination of amplitude zones B0 determined at the expiration of the second confirmation window 904, a P-wave 903 is sensed. An Asense signal 930 may be generated by the sensing circuitry. Control circuit 204 may start post-atrial sense timing control parameters (not illustrated in FIG. 11 for the sake of clarity), which may have an effective starting time at the time of the Asense signal 930 or at the time of the first sensing threshold crossing (e.g., at the starting time of the first confirmation window 902) or the expiration time of the first confirmation window 902 in various examples.

A second negative sensing threshold amplitude 916 may optionally be applied during the second confirmation window 904 in some examples. In this case, an amplitude zone for the second confirmation window 904 could be a 0 (greater than first negative sensing threshold amplitude 914), 1 (less than first negative sensing threshold amplitude 914 but greater than second negative sensing threshold amplitude 916) or 2 (less than second negative sensing threshold amplitude 916) as illustrated in FIG. 11. In an example, if the combination of amplitude zones is B0 at the expiration of the second confirmation window 904, an Asense signal 930 may be generated based on analysis of one cardiac electrical signal 901. If the combination of amplitude zones is B1 or B2 at the expiration of the second confirmation window 904, the analysis of the cardiac electrical signal 901 may be indeterminate. An analysis of the combination of amplitude zones or any other feature(s) of a second cardiac electrical signal, if available, may be performed to determine if P-wave sensing criteria are met or not as described above in conjunction with FIG. 10. If a second cardiac electrical signal is unavailable, the sensing circuitry may apply only the second positive sensing threshold amplitude 912 during the first confirmation window 902 and if it is not crossed apply only the first negative sensing threshold amplitude 914 during the second confirmation window 904. The possible combinations of amplitude zones are then B0 or B1. A P-wave is confirmed when the amplitude zone combination is B0. No cardiac event signal is sensed by the sensing circuitry if the amplitude zone combination is B1.

If the second positive sensing threshold amplitude 912 is crossed during the first confirmation window 902, the sensing circuitry may start the second confirmation window 904 and apply only the second negative sensing threshold amplitude 916 for confirming a sensed R-wave 905. The possible amplitude zone combination outcomes in this case are A1 or A2. An R-wave can be confirmed by the sensing circuitry in response to an A2 combination. A Vsense signal 932 may be generated by the sensing circuitry. Control circuit 206 may start any applicable timing control intervals having an effective starting time at the start of the first confirmation window 902, expiration of the first confirmation window 902, crossing time of the second positive sensing threshold amplitude 912, or expiration of the second confirmation window 904 in various examples. If an A1 combination is determined at the expiration of the second confirmation window 904, the sensing circuitry may not generate a sensed cardiac event signal. Any running timing control intervals may continue running without interruption. A pending ventricular pacing pulse being withheld due to a ventricular pacing interval or AV pacing interval expiring during the first confirmation window 702 or the second confirmation window 704 may be delivered by the pulse generator 202 at the expiration of the second confirmation window 704.

In some examples, after a crossing of the second positive sensing threshold 912 during the first confirmation window 902, the sensing circuitry may apply two or more sensing thresholds during the second confirmation window 904, such that the amplitude zone combination may be determined from three or more possible combinations. In this case, one combination, e.g., the A2 combination, may positively identify a sensed R-wave. Any other combination may be indeterminate. In this case, analysis of an amplitude zone combination or any other signal feature determined from a second cardiac electrical signal may be used by the sensing circuitry for positively identifying the R-wave 905 when the second negative sensing threshold 916 is not crossed.

In the example shown, the first positive sensing threshold amplitude 912 is applied outside of a running confirmation window for sensing a first threshold crossing and starting the first confirmation window 902. It is to be understood that one or more blanking periods may be applied that may extend later than the expiration of the second confirmation window, such as a post-atrial blanking period, post-ventricular blanking period, post-ventricular atrial blanking period or the like. A first threshold crossing that triggers the start of the first confirmation window may be ignored or not detected during an applicable blanking period.

As further illustrated in FIG. 11, a first negative sensing threshold amplitude 914 may optionally be applied to the cardiac electrical signal 901 outside any running confirmation windows (and any applicable blanking periods). As such, in some cases, both the first positive sensing threshold amplitude 910 and the first negative sensing threshold amplitude 914 can be applied for triggering the start of a first confirmation window 902. In response to a first negative sensing threshold crossing, the first confirmation window 902 may be started and a second negative sensing threshold amplitude 916 may be applied. The amplitude zone of the first confirmation window 902 may be determined to be C or D, either greater than or less than the second negative sensing threshold amplitude 916, respectively.

When the first confirmation window 902 expires, the second confirmation window 904 may be started. If the first confirmation window 902 is started in response to a crossing of the first negative sensing threshold amplitude 914, the sensing circuitry may apply a positive sensing threshold amplitude, e.g., the first positive sensing threshold amplitude 910 as shown and/or the second positive sensing threshold amplitude 912. In some examples, the second negative sensing threshold amplitude 916 may still be applied during the second confirmation window 904 for discriminating between a true T-wave 907 an other large negative signals that may be non-cardiac noise. In the example shown, the possible amplitude zone combinations following a first negative threshold amplitude may be C1, C2, C3, D1, D2 or D3. The sensing circuitry may positively confirm a sensed T-wave in response to a C1 amplitude zone combination. The sensing circuitry may determine that the waveform is indeterminate based on a C2, C3, D2 or D3 amplitude zone combination. If a second cardiac electrical signal is available, analysis of the second cardiac electrical signal may be relied on for positively determining the sensed waveform.

As such, as shown by FIG. 11, the amplitude and polarity of the sensing threshold(s) applied by the sensing circuitry during the first confirmation window 902 may depend on the types of cardiac event signals intended to be sensed and/or the polarity of the first sensing threshold (e.g., 910 or 914) that is crossed for triggering the start of the first confirmation window 902. The amplitude and polarity of the sensing threshold(s) applied by the sensing circuitry during the second confirmation window 904 may depend on the amplitude zone determined at the expiration of the first confirmation window 902. In some cases, a single sensing threshold, opposite in polarity of the first threshold crossing that triggered the start of the first confirmation window, is applied during the second confirmation window and may have an amplitude that depends on the amplitude zone of the first confirmation window 902. The single sensing threshold applied in the second confirmation window 904 may be used to confirm a sensed cardiac event signal or not. In other cases, multiple sensing thresholds may be applied during the first and/or second confirmation windows 902 and 904, respectively, for either confirming a sensed cardiac event signal or determining that the sensed waveform is an indiscriminate signal. In response to an indiscriminate amplitude zone combination, a second cardiac electrical signal analysis may be relied upon to either confirm a sensed cardiac event or determine that no cardiac event signal is sensed.

In some examples, a cardiac electrical signal 901 may be transmitted from the implanted pacemaker 14 to an external device, e.g., external device 20 as shown in FIG. 1. The cardiac electrical signal 901 may be displayed to a clinician or other user, e.g., in a graphical user interface of external device 20 (FIG. 1). The sensed cardiac event signals may be annotated, e.g., by Asense signal 930 and Vsense signal 932. The sensed cardiac event signals may be annotated or re-labeled by a user to enable expert truthing of sensed event signals based on user observation of the P-wave 903, R-wave 905 and/or T-wave 907. External device processor 52 and/or pacemaker control circuit 206 may adjust the cardiac event sensing criteria for one or more cardiac event signals in response to the user input.

In other examples, a user may program the amplitudes of the positive and negative sensing thresholds 910, 912, 914 and/or 916 and/or the duration of the confirmation windows 902 and 904 to provide for reliable sensing and discrimination of P-waves, R-waves and T-waves (or avoiding T-wave sensing) based on amplitude zone combinations. For instance, a user may interact with a graphical user interface presented on external device display unit 54 that includes a display similar to diagram 900. A user may enter or select a numeric value to program a given sensing threshold amplitude and polarity. In some examples, a user may click on a horizontal line representing a given sensing threshold 910, 912, 914 or 916 to slide the threshold amplitude up or down, e.g., within specified or practical limits, to select the amplitude of the respective sensing threshold.

The user may click on the horizontal line representing a given sensing threshold 910, 912, 914 or 916 to enable or disable application of the respective sensing threshold during a given confirmation window 902 or 904. Each sensing threshold 910, 912, 914 and/or 916 may be selectively enabled or disabled during the first confirmation window 902, which may be based on which first sensing threshold amplitude 910 or 914 is crossed outside of a confirmation window, to trigger the start of the first confirmation window 902. Each sensing threshold 910, 912, 914 and/or 916 may be selectively enabled or disabled during the second confirmation window 904, which may be based on the amplitude zone reached during the first confirmation window 902.

The user may individually program the time duration of the first confirmation window 902 and/or the second confirmation window 904. For example, the user may enter a numeric value or slide vertical lines left or right that represent the ending times of the confirmation windows 902 and/or 904 to adjust the duration of the respective confirmation window 902 and/or 904.

The external device processor 52 may display the cardiac electrical signal 901 for one or more heartbeats and may automatically determine possible amplitude zone combination outcomes and present the associated sensing classification outcomes in a table, e.g., similar to Table I above, based on the user programming input. In this way, a user may configure pacemaker 14 (or other medical device) to reliably sense cardiac electrical signals in a patient-specific manner. The medical device and techniques disclosed herein provide various improvements in a medical device system configured to sense cardiac event signals attendant to the depolarization and/or repolarization of cardiac tissue from one or more sensed cardiac electrical signals. The techniques disclosed herein improve the function of a medical device system in providing visual representations of cardiac event signal sensing performed by a medical device that are useful in guiding a user in programming cardiac event sensing criteria for reliably sensing and discriminating P-waves, R-waves, and/or T-waves, and in some cases non-sinus signals such as PACs and/or PVCs, from other cardiac and/or non-cardiac signals.

The techniques disclosed herein therefore provide improvements in the computer-related field of cardiac signal monitoring and cardiac therapy delivery. By providing a medical device system capable of displaying a graphical user interface according to the techniques herein, for example, the complexity and likelihood of human error in programming cardiac event sensing criteria, e.g., sensing threshold amplitudes, polarities and/or confirmation window durations, is reduced. The clinical benefit of cardiac monitoring and/or cardiac pacing to the patient can be improved by the disclosed techniques by simplifying the process of programming cardiac event sensing criteria for providing reliable cardiac event signal sensing. The techniques disclosed herein may enable selection and programming of cardiac event sensing criteria for achieving reliable sensing of P-waves, R-waves and/or T-waves with a high degree of confidence in a manner that is simplified, flexible, and patient-specific.

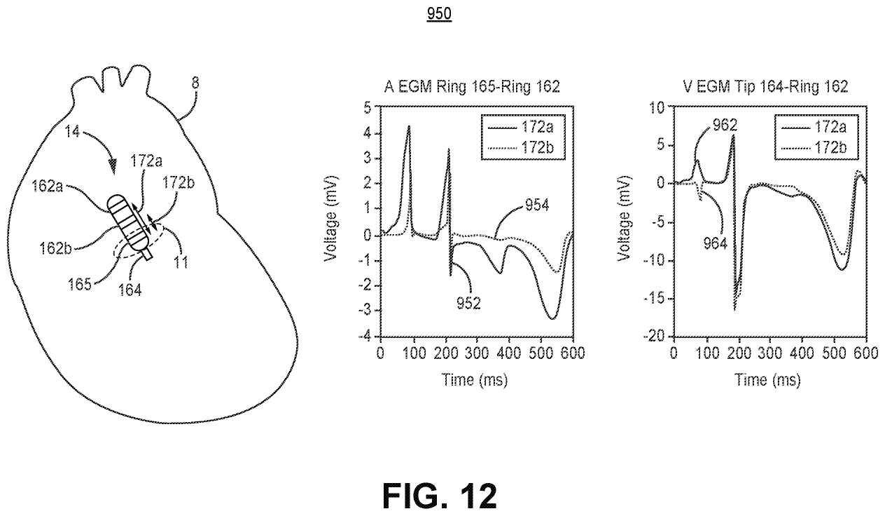

FIG. 12 is a conceptual diagram 950 illustrating possible variations in sensed cardiac electrical signals due to differences in inter-electrode spacing of a sensing electrode vector according to some examples. Pacemaker 14 is shown implanted in a patient's heart 8, e.g., in the right atrium with tip electrode 164 advanced in the Triangle of Koch 11 to a ventricular pacing site. Two different inter-electrode spacings 172a and 172b between the distal ring electrode 165 or distal tip electrode 164 and the proximal ring electrode 162a or 162b are shown in this example. The inter-electrode distance 172a to proximal ring electrode 162a is greater than the inter-electrode distance 172b to proximal ring electrode 162b from either of distal ring electrode 165 or tip electrode 164. While two proximal ring electrodes 162a and 162b are illustrated in FIG. 12, one or both electrodes 162a and/or 162b (or more proximal electrodes) may actually be present for use in sensing electrode vector(s) for sensing one or more EGM signal(s) in various examples.

An EGM signal 952 sensed between distal ring electrode 165 and proximal ring electrode 162a and an EGM signal 954 sensed between distal ring electrode 165 and proximal ring electrode 162b are shown. An EGM signal 962 sensed between tip electrode 164 and proximal ring electrode 162a and an EGM signal 964 sensed between tip electrode 164 and proximal ring electrode 162b are shown. As can be observed by EGM signals 952, 954, 962 and 964, the polarity, mono- or biphasic morphology, signal width and maximum peak amplitude and minimum peak amplitude can vary depending on the selected sensing electrode vector and inter-electrode spacing 172a and 172b. As such, the sensing electrode vector used for sensing a cardiac signal from which P-waves and/or R-waves (and optionally T-waves) are sensed by the medical device sensing circuitry may be selected to provide the greatest discrimination based on the amplitude and polarity of the programmed sensing thresholds and the duration of one or more confirmation windows according to any of the example techniques presented herein. By using programmable positive and negative sensing threshold amplitudes applied to a non-rectified cardiac signal, improvements in the reliability of sensing of P-waves, R-waves and/or T-waves without undesired oversensing can be achieved.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single circuit or unit for purposes of clarity, it should be understood that the techniques of this disclosure may be performed by a combination of units or circuits associated with, for example, a medical device.

In one or more examples, the functions described may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPLAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

1. A medical device comprising:
a therapy delivery circuit configured to deliver a therapy;
sensing circuitry configured to:
sense a first cardiac signal;
without rectification of the first cardiac signal, apply a first sensing threshold amplitude having a first polarity to the first cardiac signal;
in response to the first cardiac signal crossing the first sensing threshold amplitude, apply a second sensing threshold amplitude to the first cardiac signal, the second sensing threshold amplitude having a second polarity opposite the first polarity;
determine when a requirement relating to the second sensing threshold amplitude is met by the first cardiac signal; and
confirm a sensed first cardiac event signal corresponding to a depolarization of a first heart chamber in response to at least the first cardiac signal crossing the first sensing threshold amplitude and meeting the requirement relating to the second sensing threshold amplitude; and
control circuitry configured to:
start a first timing control interval in response to the confirmed sensed first cardiac event signal; and
control at least one of the sensing circuitry or the therapy delivery circuit according to the first timing control interval.

2. The medical device of claim 1 wherein the sensing circuitry is further configured to:
start a first confirmation window in response to the first cardiac signal crossing the first sensing threshold amplitude; and
determine that the requirement relating to the second sensing threshold amplitude is met by the first cardiac signal being sensed during the first confirmation window.

3. The medical device of claim 1 wherein the sensing circuitry is configured to determine that the requirement relating to the second sensing threshold amplitude is met by the first cardiac signal by determining that the first cardiac signal does not cross the second sensing threshold amplitude.

4. The medical device of claim 2 wherein the sensing circuitry is further configured to:
sense a second cardiac signal;
apply a third sensing threshold amplitude to the second cardiac signal;
start a second confirmation window in response to the second cardiac signal crossing the third sensing threshold amplitude; and
determine that the second confirmation window is running during a portion of the first confirmation window; and
in response to the second confirmation window running during a portion of the first confirmation window, withhold confirming the sensed first cardiac event signal.

5. The medical device of claim 4 wherein:
the sensing circuitry is further configured to:
apply a fourth sensing threshold amplitude to the second cardiac signal sensed during the second confirmation window, the fourth sensing threshold amplitude having an opposite polarity from the third sensing threshold amplitude;
determine that the second cardiac signal meets a requirement relating to the fourth sensing threshold amplitude having a different polarity than the third sensing threshold amplitude; and
confirm a sensed second cardiac event signal corresponding to a depolarization of a second heart chamber different than the first heart chamber in response to the second cardiac signal crossing the third sensing threshold amplitude and meeting the requirement relating to the fourth sensing threshold amplitude; and
the control circuitry further configured to start a second timing control interval in response to the confirmed sensed second cardiac event signal.

6. (canceled)

7. The medical device of claim 5 wherein the control circuitry is configured to start the second timing control interval by starting a pacing interval; and
the therapy delivery circuit configured to generate a pacing pulse upon expiration of the pacing interval.

8. The medical device of claim 1 wherein the sensing circuitry is further configured to:
in response to the first cardiac signal crossing the first sensing threshold amplitude, start a first confirmation window;
apply a third sensing threshold amplitude to the first cardiac signal during the first confirmation window;
start a second confirmation window upon expiration of the first confirmation window;
apply the second sensing threshold amplitude having the second polarity opposite the first polarity during the second confirmation window;
determine a first amplitude zone relative to the first sensing threshold amplitude and the third sensing threshold amplitude;
determine a second amplitude zone relative to the second sensing threshold amplitude; and
determine when the requirement relating to the second sensing threshold amplitude is met based on the first amplitude zone and the second amplitude zone.

9. The medical device of claim 8 wherein the sensing circuitry is further configured to select the second sensing threshold amplitude that is applied during the second confirmation window based on the first amplitude zone.

10. The medical device of claim 8 wherein the sensing circuitry is further configured to, in response to determining that the requirement relating to the second sensing threshold amplitude is not met, confirm a sensed second cardiac event signal corresponding to a second heart chamber based on the first amplitude zone and the second amplitude zone.

11. (canceled)

12. The medical device of claim 1 wherein the control circuitry is further configured to start the first timing control interval by starting at least one of:
a blanking period;
a refractory period; and
a pacing interval.

13. The medical device of claim 1 wherein the control circuitry is further configured to:
start the first timing control interval by starting at least a first pacing interval in response to the confirmed sensed first cardiac event signal; and
determine that the first pacing interval is expired; and
the therapy delivery circuit is further configured to deliver the therapy according to the first timing control interval by generating a pacing pulse in response to the first pacing interval being expired.

14. The medical device of claim 13, wherein:
the sensing circuitry is further configured to confirm the sensed first cardiac event as an atrial event signal; and
the control circuitry is configured to start at least the first pacing interval by starting an atrioventricular pacing interval; and
the therapy delivery circuit is configured to generate the pacing pulse by generating a ventricular pacing pulse upon expiration of the atrioventricular pacing interval.

15. The medical device of claim 1 further comprising:
a housing enclosing the sensing circuitry and the control circuitry; and
at least one leadless, housing-based tissue piercing electrode coupled to the therapy delivery circuit,
wherein the therapy delivery circuit being configured to deliver the therapy by delivering a pacing pulse to a conduction system of a patient's heart via the housing-based tissue piercing electrode upon expiration of the first timing control interval.

16. A method comprising:
sensing a first cardiac signal;
without rectifying the first cardiac signal, applying a first sensing threshold amplitude having a first polarity to the first cardiac signal;
in response to the first cardiac signal crossing the first sensing threshold amplitude, applying a second sensing threshold amplitude to the first cardiac signal, the second sensing threshold amplitude having a second polarity opposite the first polarity;
determining when a requirement relating to the second sensing threshold amplitude is met by the first cardiac signal;
confirming a sensed first cardiac event signal corresponding to a depolarization of a first heart chamber in response to at least the first cardiac signal crossing the first threshold amplitude and meeting the requirement relating to the second sensing threshold amplitude;
starting a first timing control interval in response to the confirmed sensed first cardiac event signal; and
controlling at least one of sensing of the first cardiac signal or delivery of a therapy according to the first timing control interval.

17. The method of claim 16 further comprising:
starting a first confirmation window in response to the first cardiac signal crossing the first sensing threshold amplitude; and
determining that the requirement relating to the second sensing threshold amplitude is met by the first cardiac signal sensed during the first confirmation window.

18. The method of claim 16 further comprising determining that the requirement relating to the second sensing threshold amplitude is met by the first cardiac signal by determining that the first cardiac signal does not cross the second sensing threshold amplitude.

19. The method of claim 17 further comprising:
sensing a second cardiac signal;
applying a third sensing threshold amplitude to the second cardiac signal;
starting a second confirmation window in response to the second cardiac signal crossing the third sensing threshold amplitude;
determining that the second confirmation window is running during a portion of the first confirmation window; and
in response to the second confirmation window running during a portion of the first confirmation window, withholding confirming the sensed first cardiac event signal.

20. The method of claim 19 further comprising:
applying a fourth sensing threshold amplitude to the second cardiac signal sensed during the second confirmation window, the fourth sensing threshold amplitude having an opposite polarity from the third sensing threshold amplitude;
determining that the second cardiac signal meets a requirement relating to the fourth sensing threshold amplitude having a different polarity than the third sensing threshold amplitude;
confirming a sensed second cardiac event signal corresponding to a depolarization of a second heart chamber different than the first heart chamber in response to the second cardiac signal crossing the third sensing threshold amplitude and meeting the requirement relating to the fourth sensing threshold amplitude; and
starting a second timing control interval in response to the confirmed sensed second cardiac event signal.

21. The method of claim 19 further comprising:
starting the second timing control interval by starting a pacing interval; and
generating a pacing pulse upon expiration of the pacing interval.

22. The method of claim 16 further comprising:
starting the first timing control interval by starting a first pacing interval and starting at least one of:
a blanking period applied to the first cardiac signal; and
a refractory period applied to the first cardiac signal; and
controlling the delivery of the therapy according to the first timing control interval by delivering a cardiac pacing pulse in response to the pacing interval expiring.
